(19) United States
(12) Patent Application Publication
(10) Pub. No.: US 2025/0160907 A1
(43) Pub. Date: May 22, 2025

(54) SPINAL FIXATION ACCESS AND DELIVERY SYSTEM

(71) Applicant: PROVIDENCE MEDICAL TECHNOLOGY, INC., Pleasanton, CA (US)

(72) Inventors: Shigeru Tanaka, Half Moon Bay, CA (US); Christopher U. Phan, Dublin, CA (US); Christopher Lambert, Concord, CA (US); Nicholas Domek, Walnut Creek, CA (US); Bon Champ, Capbell, CA (US); Edward Liou, Pleasanton, CA (US)

(21) Appl. No.: 19/024,555

(22) Filed: Jan. 16, 2025

Related U.S. Application Data
(63) Continuation of application No. 18/380,371, filed on Oct. 16, 2023.
Application No. 18/380,371 is a continuation of application No. 16/614,888, filed on Nov. 19, 2019, now Pat. No. 11,871,968.
(60) Provisional application No. 62/508,434, filed on May 19, 2017.

(51) Int. Cl. A61B 17/70; A61B 17/02; A61B 17/16; A61B 90/00

(52) U.S. Cl. CPC A61B 17/7064; A61B 17/7032; A61B 17/7037; A61B 17/7076; A61B 17/7086; A61B 2017/0256; A61B 17/1671; A61B 2090/036

(57) ABSTRACT

A spinal fixation access and delivery system is disclosed. In some aspects, the system is used to access the cervical facet joint via a posterior access approach. The system may include an access device including a body having a proximal portion and a distal portion and a chamfered or beveled end feature positioned at the distal portion of the body and configured for insertion at the cervical facet joint. The system may further include a guide device having access device engagement features and a spinal fixation member. The access device includes at least one guide device receiving feature complementary to or keyed to the engagement feature of the guide device.

### CROSS-REFERENCE TO RELATED APPLICATION

This application is a continuation of U.S. patent application Ser. No. 18/380,371, filed Oct. 16, 2023, which is a continuation of U.S. patent application Ser. No. 16/614,888, filed Nov. 19, 2019, now U.S. Pat. No. 11,871,968, issued Jan. 16, 2024, which is a national stage application of International Patent Application No. PCT/US2018/033505, filed May 18, 2018, which claims priority to U.S. Provisional Patent Application No. 62/508,434, filed May 19, 2017, all titled “SPINAL FIXATION ACCESS AND DELIVERY SYSTEM.” The full disclosures of the above-listed patent applications are hereby incorporated by reference in their entireties and for all purposes.

### FIELD

This application is directed to medical devices and methods. More specifically, the application is directed to devices and methods related to spinal fixation to provide stability to the spine and promote spinal fusion.

### BACKGROUND

Chronic back problems are one of the most common causes of pain and disability in the United States and other developed countries. According to at least one estimate, spinal fusion procedures, in which two adjacent vertebrae are fused together using plates, screws and other implants, are the most commonly performed surgical procedures in the United States. Spinal fusion is often performed in an attempt to increase space between the two adjacent vertebrae being operated on (known as spinal distraction) and to thus prevent impingement of the spinal cord or nerve roots branching from the spinal cord and passing through openings in the vertebral column. Unfortunately, most techniques and devices used for performing spinal fusion are relatively invasive and involve a number of risks and difficult recovery and rehabilitation.

Posterior spinal fusion is one method of surgical intervention. However, in order to provide direct visualization of the target area, it requires extensive dissection of muscles and ligaments. This dissection causes acute and chronic soft tissue pain syndrome. Acutely, patients are typically hospitalized for three to four days for pain control that requires IV narcotics. Long-term, patients frequently have persistent pain due to the extensive nature of the dissection. This is compared to one-day hospitalization for anterior approaches that do not require any muscle or soft tissue dissection. In some cases, soft tissues may not return to anatomic position and may be permanently deformed. Persistent pain after posterior surgical approaches is referred to as post-laminectomy syndrome.

Therefore, since it is considered less traumatic to the patient, anterior spinal fusion surgery has generally been preferred over posterior fusion surgery. However, posterior approaches to the cervical spine do have some advantages over anterior approaches.

Lateral mass or pedicle screw fixation provides more rigid fixation of the cervical spine than anterior plates, interbody devices and interspinous wiring. It is often used for traumatic instability, but it has also been used for degenerative conditions. Despite providing good results, lateral mass fixation is often avoided because of the morbidity of the soft tissue dissection, as noted above.

Therefore, a need exists for alternative devices and methods for fixation of the spine following surgery for fusion of adjacent vertebrae. Ideally, these devices, systems and methods would allow for minimally invasive or less invasive access and fixation that many of the currently available techniques do not provide. For example, it may be advantageous to have devices, systems and methods that use a posterior approach for accessing the spine. At least some of these objectives will be met by the embodiments described herein.

### BRIEF SUMMARY

The various embodiments described herein provide devices, systems and methods for accessing the cervical spine via a posterior approach and delivering or providing a spinal fixation device for fixation of the cervical spine. The embodiments described below generally include an access and delivery system through which or along which one or more spinal fixation devices may be advanced. The access devices described herein generally include a distal end that can be inserted into a cervical facet. Once inserted into the facet, the access device can be used as a point of stabilization.

A cervical facet joint access device for accessing the cervical facet joint via a posterior access approach is disclosed. In some aspects, the device includes a body having a proximal portion and a distal portion and a chamfered or beveled end feature positioned at the distal portion of the body and configured for insertion at the cervical facet joint. In some aspects, the chamfered or beveled end feature is offset from the body. In some aspects, the body is an elongated body having opposing top and bottom faces, opposing side faces and opposing end faces and the chamfered or beveled end feature is offset from the body and positioned on one of the opposing side faces. In some aspects, the body has a rectangular shaped cross section. In some aspects, the proximal portion has a first height and the distal portion has a second height and the first height is greater than the second height. In some aspects, the body is an elongated tubular body. In some aspects, the chamfered or beveled end feature is positioned at an end of the distal portion. In some aspects, the end further includes a stop adapted to abut a posterior edge of the facet joint. The stop may include a raised or protruding feature adapted to engage the facet joint. In some aspects, the end feature includes an expandable anchor held in a closed position via a detent feature and opened by actuation of an internal rod to pivot the arms of the end feature into an open position. In some aspects, the end feature includes an expandable member held in a closed position for delivery and expanded into an open position by actuation of an internal rod. In some aspects, the end feature comprises an articulating tip. In some aspects, the body is a tubular body adapted to receive other surgical instruments for spinal fixation.

A spinal fixation access and delivery system for accessing the cervical facet joint via a posterior access approach is disclosed. In some aspects, the system includes an access device including a body having a proximal portion and a distal portion and a chamfered or beveled end feature positioned at the distal portion of the body and configured for insertion at the cervical facet joint. The system may further include a guide device having access device engagement features and a spinal fixation member. The access device includes at least one guide device receiving feature complementary to or keyed to the engagement feature of the guide device.

The access device may be the access device as disclosed herein. In some aspects, the chamfered or beveled end feature of the access device is offset from the body. In some aspects, the body is an elongated body having opposing top and bottom faces, opposing side faces and opposing end faces and the chamfered or beveled end feature is offset from the body and positioned on one of the opposing side faces. In some aspects, the body has a rectangular shaped cross section. In some aspects, the proximal portion has a first height and the distal portion has a second height and the first height is greater than the second height. In some aspects, the body is an elongated tubular body. In some aspects, the chamfered or beveled end feature is positioned at an end of the distal portion. In some aspects, the end further includes a stop adapted to abut a posterior edge of the facet joint. The stop may include a raised or protruding feature adapted to engage the facet joint. In some aspects, the end feature includes an expandable anchor held in a closed position via a detent feature and opened by actuation of an internal rod to pivot the arms of the end feature into an open position. In some aspects, the end feature includes an expandable member held in a closed position for delivery and expanded into an open position by actuation of an internal rod. In some aspects, the end feature comprises an articulating tip. In some aspects, the body is a tubular body adapted to receive other surgical instruments for spinal fixation.

In some aspects, the device engagement features are selected from a protrusion, a notch or a recess. In some aspects, the system may further include a decortication tool. A portion of the decortication tool may optionally include a burr, a rasp or one or more teeth.

A spinal fixation access and delivery system for accessing the cervical facet joint via a posterior access approach is disclosed. In some aspects, the system includes an access device, a spinal fixation member, and a guide device having at least one spinal fixation member engagement feature. The spinal fixation member includes at least one guide device receiving feature complementary to or keyed to the engagement feature of the guide device. In some aspects, the spinal fixation member is a tower or a polyaxial screw with a tower feature. In some aspects, the at least one spinal fixation member engagement feature is a generally cylindrical body protruding from an outer surface of the guide device and generally extending the length of the guide device. The access device may be the access device as disclosed herein.

In some aspects, the chamfered or beveled end feature of the access device is offset from the body. In some aspects, the body is an elongated body having opposing top and bottom faces, opposing side faces and opposing end faces and the chamfered or beveled end feature is offset from the body and positioned on one of the opposing side faces. In some aspects, the body has a rectangular shaped cross section. In some aspects, the proximal portion has a first height and the distal portion has a second height and the first height is greater than the second height. In some aspects, the body is an elongated tubular body. In some aspects, the chamfered or beveled end feature is positioned at an end of the distal portion. In some aspects, the end further includes a stop adapted to abut a posterior edge of the facet joint. The stop may include a raised or protruding feature adapted to engage the facet joint. In some aspects, the end feature includes an expandable anchor held in a closed position via a detent feature and opened by actuation of an internal rod to pivot the arms of the end feature into an open position. In some aspects, the end feature includes an expandable member held in a closed position for delivery and expanded into an open position by actuation of an internal rod. In some aspects, the end feature comprises an articulating tip. In some aspects, the body is a tubular body adapted to receive other surgical instruments for spinal fixation.

A cervical spinal fixation member is disclosed. In some aspects, the spinal fixation member includes an elongated tubular body having a length extending between a distal and a proximal end, a rod receiving slot defined in at least a portion of the length of the tubular body; and a polyaxial screw. The spinal fixation member may further include a rod. In some aspects, the rod receiving slot further includes an opening defined in the outer circumference of the elongated tubular body. In some aspects, the rod receiving slot extends only a portion of the length of the elongated body and the elongated body is solid for the remainder of the length.

These and other aspects and embodiments will be described in further detail below, in reference to the attached drawing figures.

### BRIEF DESCRIPTION OF DRAWINGS

FIG. 1 is a lateral view of the C5 and C6 (cervical) vertebrae.

FIG. 2A is a perspective view of an access device according to the present disclosure.

FIGS. 2B-2E are various views of the access device of FIG. 2A in use.

FIGS. 3A-3C are perspective, top and partial rear views of another access device according to the present disclosure.

FIG. 3D is a posterior view of the access devices of both FIG. 2A and FIG. 3A in use.

FIGS. 4A-4C are partial perspective views of an access device according to the present disclosure having a stop feature.

FIGS. 4D-4G are various views of the access devices of FIGS. 4A-4C in use.

FIGS. 5A-5D are side and isometric views of an access device according to the present disclosure having an expandable end feature, where FIGS. 5A and 5C show a closed position and FIGS. 5B and 5D show an open position.

FIGS. 5E-5F show the access device of FIGS. 5A-5D in use.

FIGS. 6A-6D are side and isometric views of an access device according to the present disclosure having an expandable end feature, where FIGS. 6A and 6C show a closed position and FIGS. 6B and 6D show an open position.

FIGS. 6E-6F show the access device of FIGS. 6A-6D in use.

FIGS. 7A-7B are top and lateral views of an access device according to the present disclosure having an end feature with a rigid tip.

FIG. 7C is the device of FIG. 7A-7B in use.

FIGS. 8A-8B are top and isometric views of an access device according to the present disclosure having an end feature with an articulating tip.

FIGS. 8C-8F are enlarged views of the tip of FIGS. 8A-B, in unlocked (FIGS. 8C-D) and locked (FIGS. 8E-F) positions.

FIGS. 8G-H are cross-section views of the tip of FIGS. 8C-8F.

FIGS. 9A-9B are top and isometric views of an access device according to the present disclosure having an end feature with an articulating tip.

FIGS. 9C-9E are enlarged views of the tip of FIGS. 9A-B, in unlocked (FIGS. 9C-D) and locked (FIG. 9E) positions.

FIGS. 9F-G are cross-section views of the tip of FIGS. 9C-9E.

FIGS. 10A-10B are top and isometric views of an access device according to the present disclosure having an end feature with an articulating tip.

FIGS. 10C-10D are enlarged views of the tip of FIGS. 10A-B, in unlocked (FIG. 10C) and locked (FIG. 10D) positions.

FIGS. 10E-F are cross-section views of the tip of FIGS. 10C-D.

FIGS. 11A-11B are top and isometric views of an access device according to the present disclosure having an end feature with an articulating tip.

FIGS. 11C-11H are enlarged or transparent views of the tip of FIGS. 11A-B, in unlocked (FIGS. 11C, 11E-F) and locked (FIGS. 11D, 11G-H) positions.

FIGS. 12A-12B are top and isometric views of an access device according to the present disclosure having an end feature with an articulating tip.

FIGS. 12C-12H are enlarged or transparent views of the tip of FIGS. 12A-B, in unlocked (FIGS. 12C, 12E-F) and locked (FIGS. 12D, 12G-H) positions.

FIGS. 13A-13F are a combined access and guide system according to aspects of the present disclosure.

FIGS. 14A-14G illustrate an access and delivery system according to aspects of the present disclosure.

FIGS. 14H-14S illustrate the system of FIGS. 14A-14G in use.

FIGS. 15A-15G illustrate an access and delivery system according to aspects of the present disclosure.

FIGS. 15H-15R illustrate the system of FIGS. 15A-15G in use.

FIGS. 16A-16C illustrate an access and delivery system according to aspects of the present disclosure which may be further stabilized by engagement with a fixation device.

FIGS. 17A-17F illustrate a portion of an access and delivery system according to aspects of the present disclosure which may be further stabilized by engagement with a fixation device.

FIGS. 18A-H are cross-sections of various embodiments of a portion of an access and delivery system according to aspects of the present disclosure which may be further stabilized by engagement with a fixation device.

FIGS. 19A-19E are various views of a temporary locking screw that may be used with the system of FIG. 17.

FIGS. 19F-19Q show the screw and system of FIGS. 17 and 19A-19E in use.

FIG. 20A is a fixation device for use with the devices and systems of the present disclosure.

FIGS. 20B-20E show the fixation device of FIG. 20A in use.

### DETAILED DESCRIPTION

Spinal stenosis reflects a narrowing of one or more areas of the spine, often in the upper or lower back. This narrowing can put pressure on the spinal cord or on the nerves that branch out from the compressed areas. Individual vertebrae of the spine are positioned relative to each other, and their separation is maintained by discs separating main vertebral bodies and by capsules positioned within facet joints. The discs and capsules are separated from the bone of their respective joints by cartilage. Spinal stenosis is often indicative of degeneration of a disc, a capsule, or the cartilage in a joint, which leads to a compression of the joints and the narrowing mentioned.

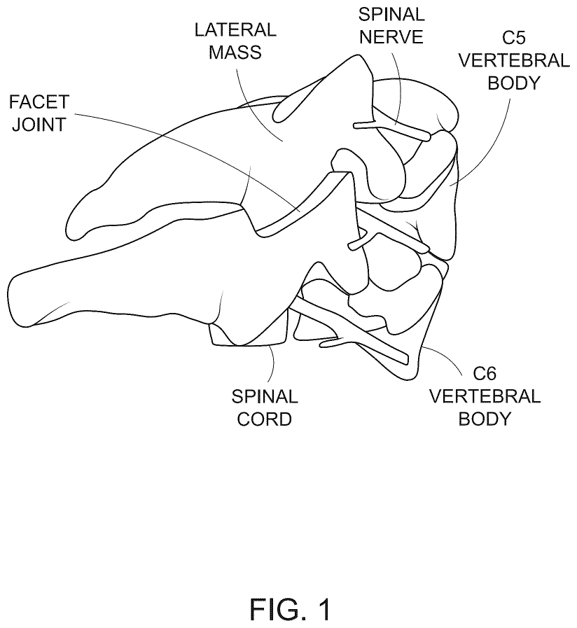

Options for distracting two adjacent vertebrae of a spine, such as the cervical vertebrae shown in FIG. 1, in an effort to ameliorate spinal stenosis, are varied and may include the use of implants, or cages and screws. In many cases, it may be possible to insert a facet joint implant into a facet joint by itself and, due to the design of the implant, do nothing further to secure the implant within the joint. In other words, the shape, size, surface features and overall configuration of the implant may cause it to remain securely within the facet joint without further attachment devices required. In some cases however, and in general for overall safety of a facet joint distraction procedure, it may be advantageous to use one or more additional devices, such as screws, to help secure the facet joint implant to one or both of the adjacent vertebrae that form the joint with a screw.

Still further, it may be advantageous to provide additional stabilization to the fusion site with the use of a pedicle screw and/or a lateral mass screw as a supplement to spinal fusion surgery. A pedicle screw or lateral mass screw are types of bone screws designed for insertion into the pedicle or lateral mass of a vertebra, respectively. The screws are inserted in adjacent vertebrae (e.g., consecutive spine segments such as C5 and C6) and then connected via a rod to prevent motion at those segments that are being fused. In this way, the screws act as anchor points for the rod and provide additional stability to fusion site to promote better fusion. Post-fusion, the rods and screws can be removed.

Described herein are devices, systems and methods for accessing the cervical spine via a posterior approach and introducing, implanting and/or securing a spinal fixation device, such as screws and rods, in the spine. Accessing the cervical spine via a posterior approach utilizes minimally invasive or less invasive techniques. Aspects described below generally include an access tool and a guide tool, through which or along which one or more spinal fixation devices may be advanced. In addition, a separate decortication tool may be advanced through or with the help of the guide tool. The decortication tool may include a burr or a rasp or teeth or other bone roughening feature for preparing the bone surface prior to insertion of the spinal fixation device.

In use, the surgeon advances the access tool into the facet joint through a minimally invasive or less invasive incision. Once anchored into place, this access tool provides a fixed point deep in the spine that is then used as a marker to advance drills, awls, plates, rods and screws, and other instruments to the cervical spine from a posterior approach without direct visualization. Such an approach with the disclosed devices prevents instruments from slipping off the spine or drills catching soft tissue and skidding out of control. In addition, the cervical facet has a fixed anatomical relationship to lateral mass bone consistent in most, if not all, patients. Instruments can be advanced over, along or about the access tool to reliable landmarks on or at the lateral mass without direct visualization. For example, to aid in spinal fixation, lateral mass screws or pedicle screws may be inserted with the help of a guide tool.

Turning now to the figures, the access tool or access device may also be referred to as an access chisel or an access anchor. The access device is advanced to the facet joint defined between adjacent vertebra, such as the vertebra of the cervical spine. The device provides access to the facet joint and surrounding anatomical structures, such as the lateral mass and the pedicle.

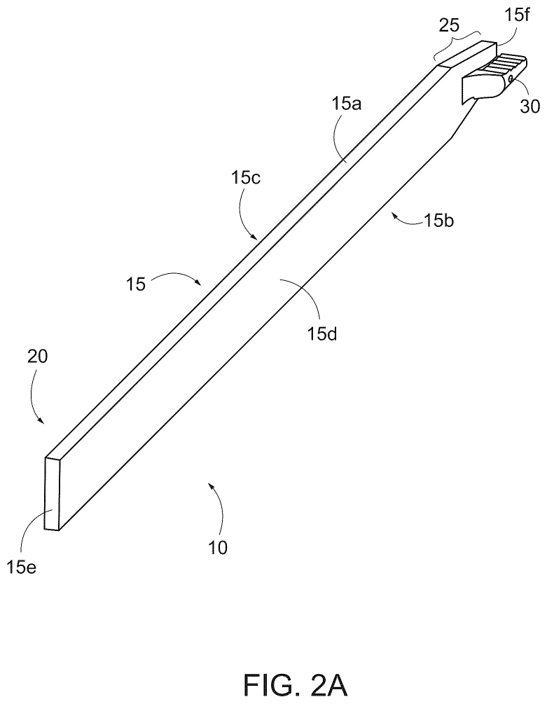

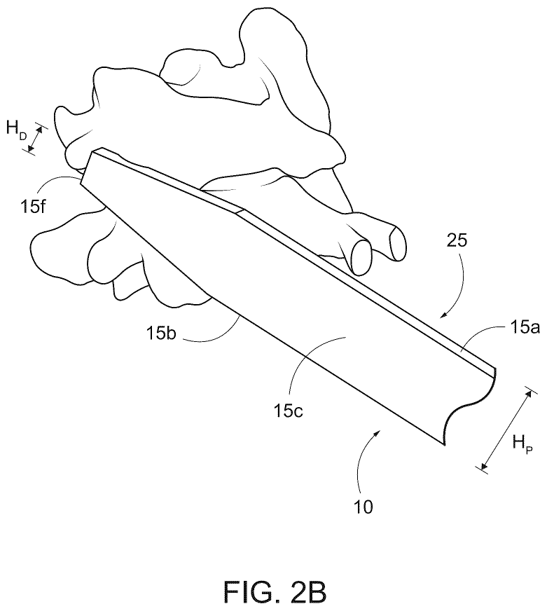

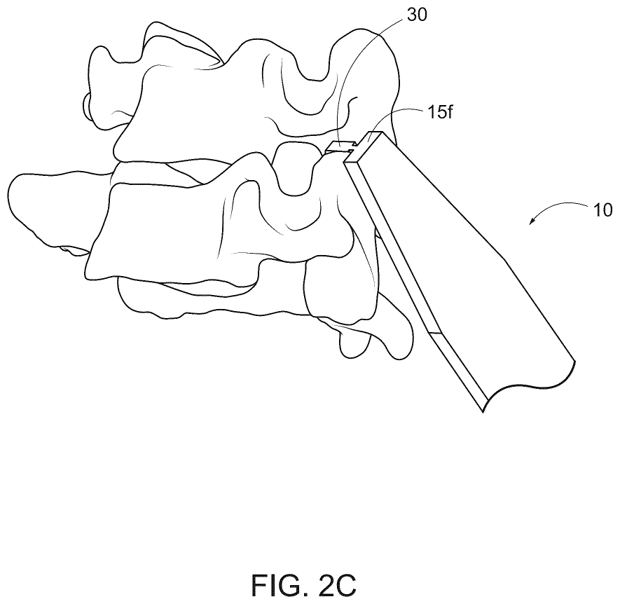

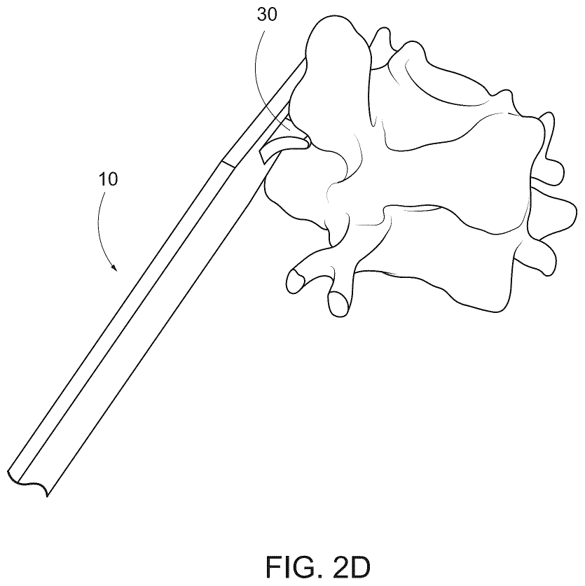

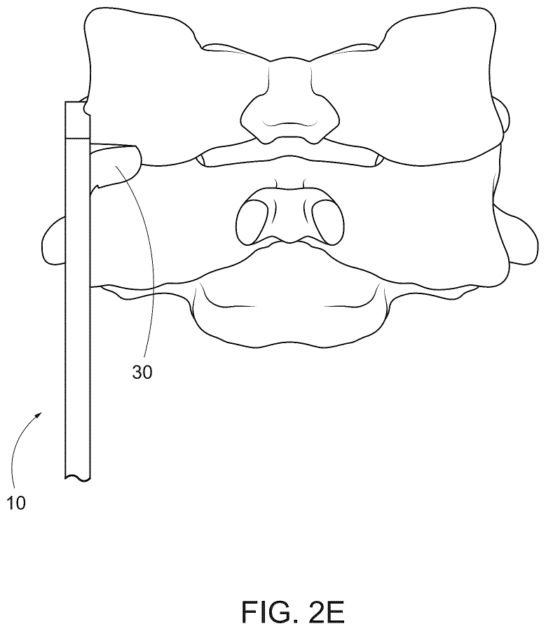

Referring now to FIGS. 2A-12H, the access device 10 includes a body 15 having a proximal portion 20 and a distal portion 25. The distal portion 25 further includes a chamfered or beveled end feature 30. The access device may be made of titanium, stainless steel, polycarbonate, or any other metal, metal alloy or polymer of sufficient strength.

In some aspects, as depicted in FIGS. 2A-2E, the body 15 of the access device 10 is an elongated body having opposing top and bottom faces 15a, 15b, opposing side faces 15c, 15d and opposing end faces 15e, 15f. The body has a generally rectangular cross-section at the proximal portion 20 which extends towards the distal portion 25. At the distal portion, the elongated body steps down or narrows relative to the proximal portion. That is, and with reference to FIG. 2B, a height Hp of the proximal portion is greater than a height Hp of the distal portion of the body 15. The proximal portion is configured to receive a removable handle (not shown) for ease of the user in manipulation of the device 10. The distal portion 25 further includes a chamfered or beveled end feature 30 positioned on a side face 15d of the elongated body. That is, the end feature 30 is offset relative to the body.

As can be understood from FIGS. 2B-2D, which illustrate the device 10 inserted at or about the facet joint, the elongated body may be vertically oriented to give vertical rigidity in the cranial-caudal direction to help resist slipping of the pedicle or lateral mass screw (insertion described in more detail below). Further, the elongated body is used to reference off the lateral aspect of the lateral mass (see FIGS. 2D-2E) and such a shape may advantageously provide for an unobstructed approach to the lateral mass.

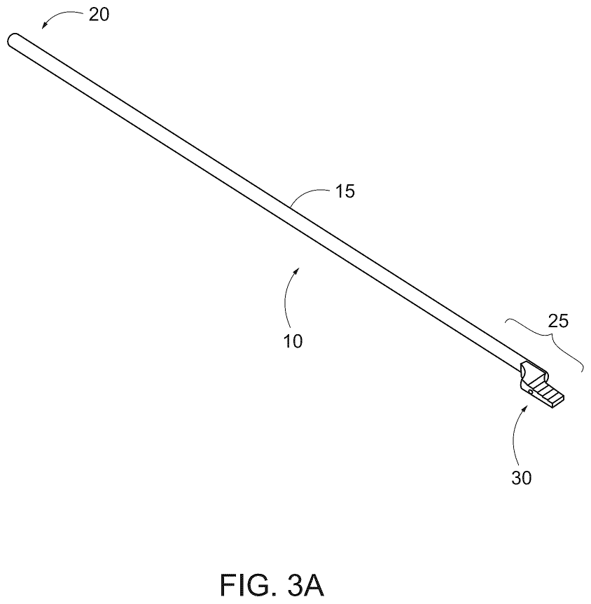

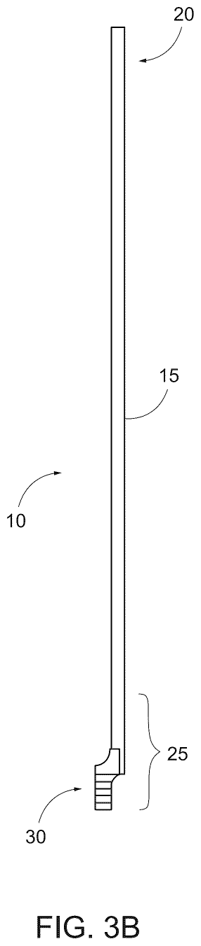

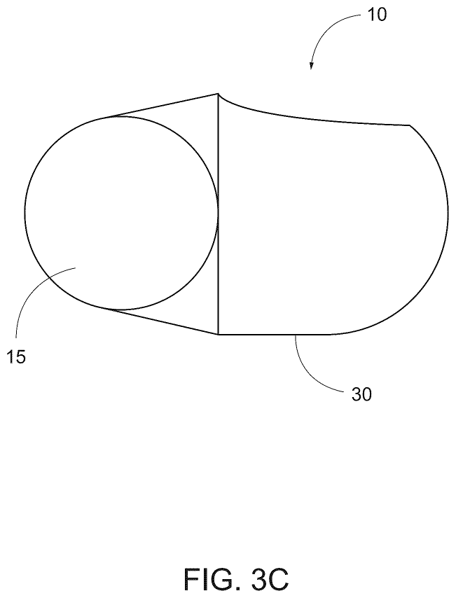

As depicted in FIGS. 3A-3C, in some aspects, the access device 10 includes the body 15 which may be an elongated tubular body having a proximal portion 20 and a distal portion 25. The distal portion 25 further includes a chamfered or beveled end feature 30 positioned on a side of the elongated body. That is, the end feature 30 is offset relative to the body. The tubular body has a low profile and may be used to slidingly receive other instruments along its body.

FIG. 3D depicts both the access device of FIGS. 2A-2E and the access device of FIGS. 3A-3C, and illustrates an asymmetrical chamfered or beveled end feature which provides a device that is offset from the axial approach to the joint space.

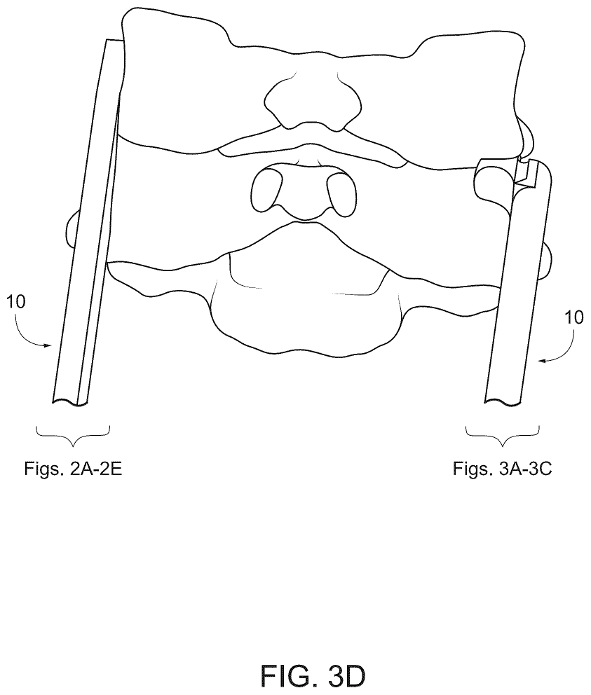

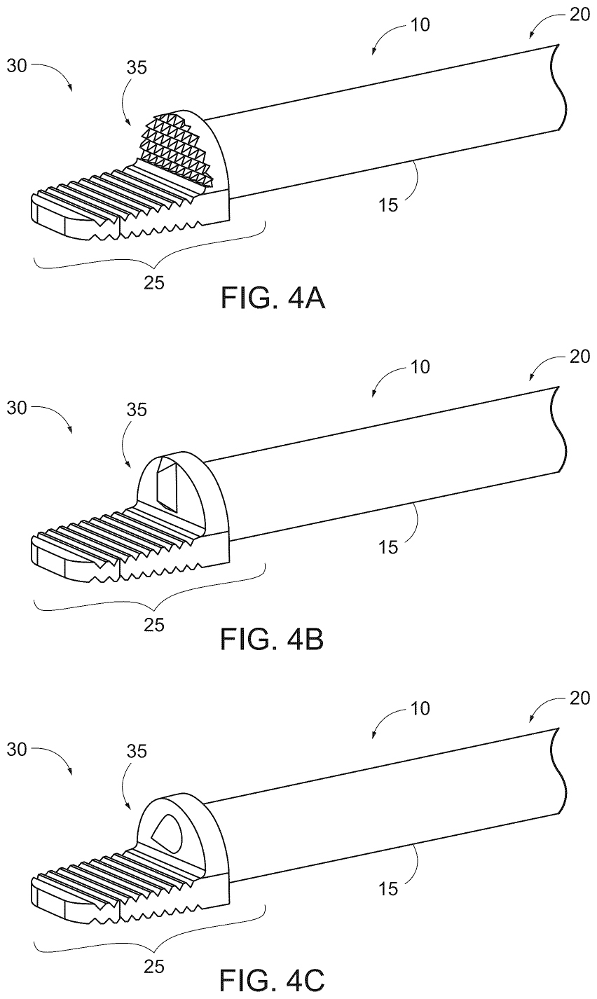

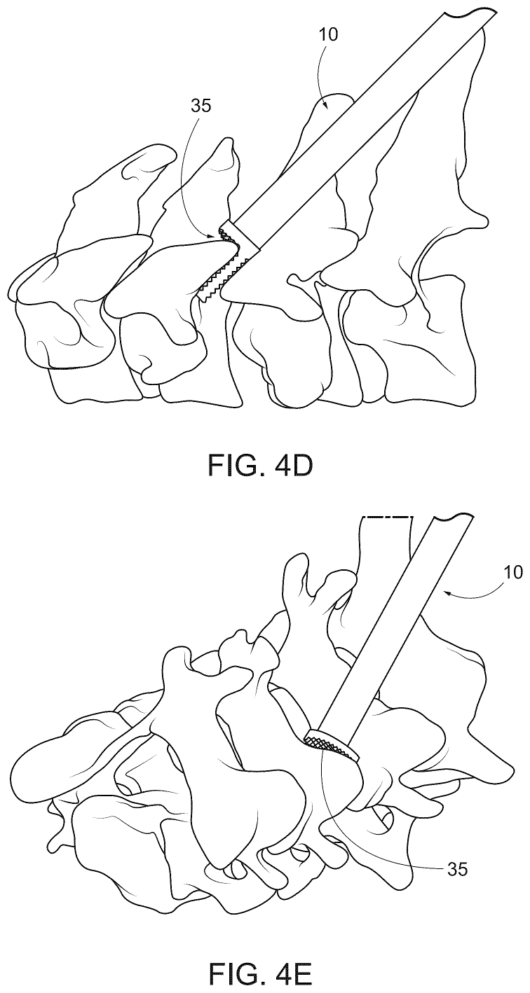

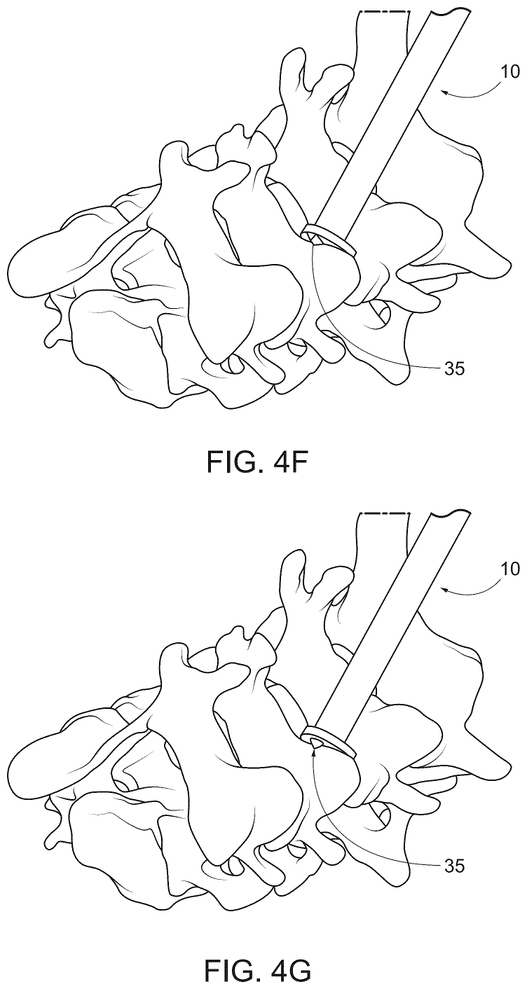

In some aspects, and as can be understood from FIGS. 4A-4G, the access device 10 includes the body 15 which may be an elongated tubular body having a proximal portion 20 and a distal portion 25. The distal portion 25 further includes a chamfered or beveled end feature 30 positioned at an end of the distal portion of the access device. The end feature 30 further includes a stop feature 35 configured to abut the posterior edge of the facet joint. The stop feature 35 may include a raised or protruding feature such as a single spike (FIG. 4C) or a plurality of spikes or teeth (FIG. 4A), such as waffle pattern spikes, or a keel (FIG. 4B). The raised or protruding feature engages the facet joint to anchor the device 10 into the facet joint. FIGS. 4D-4G illustrate the stop features depicted in FIGS. 4A-4C in use, where the device 10 is inserted in the facet joint, the stop feature 35 abuts the posterior edge of the facet joint and the raised or protruding feature engages the joint to anchor the device 10.

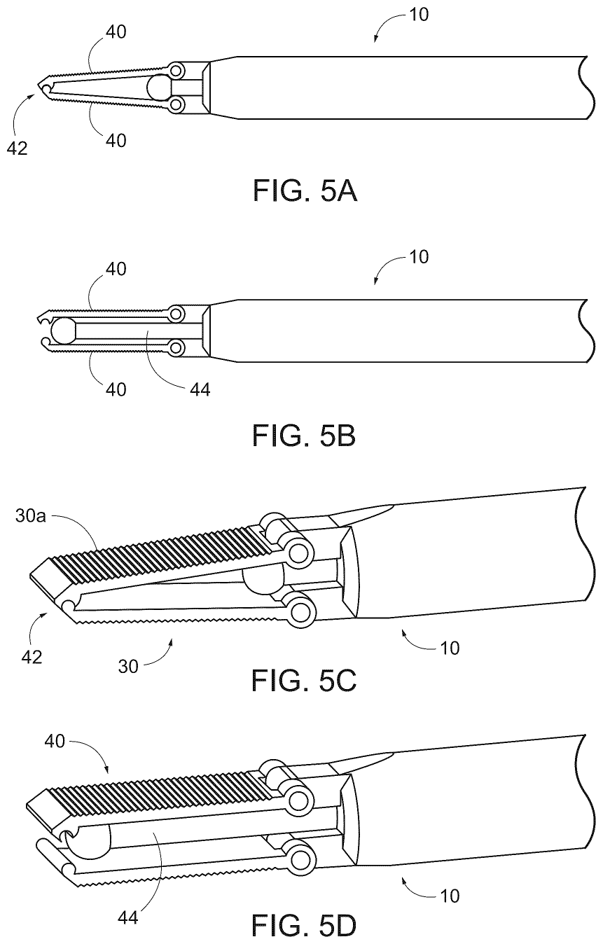

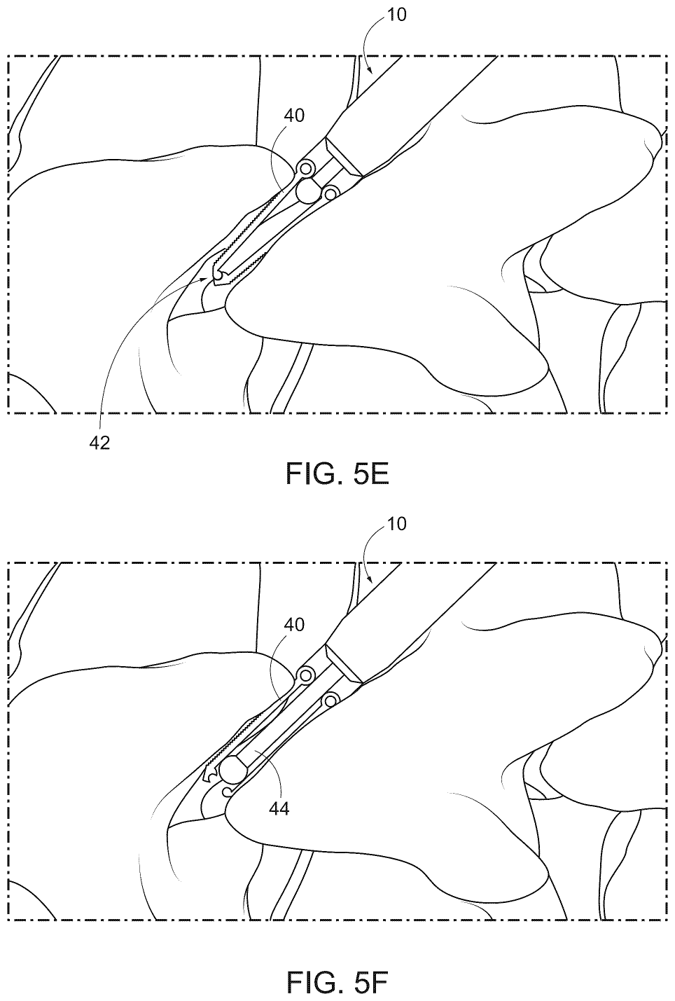

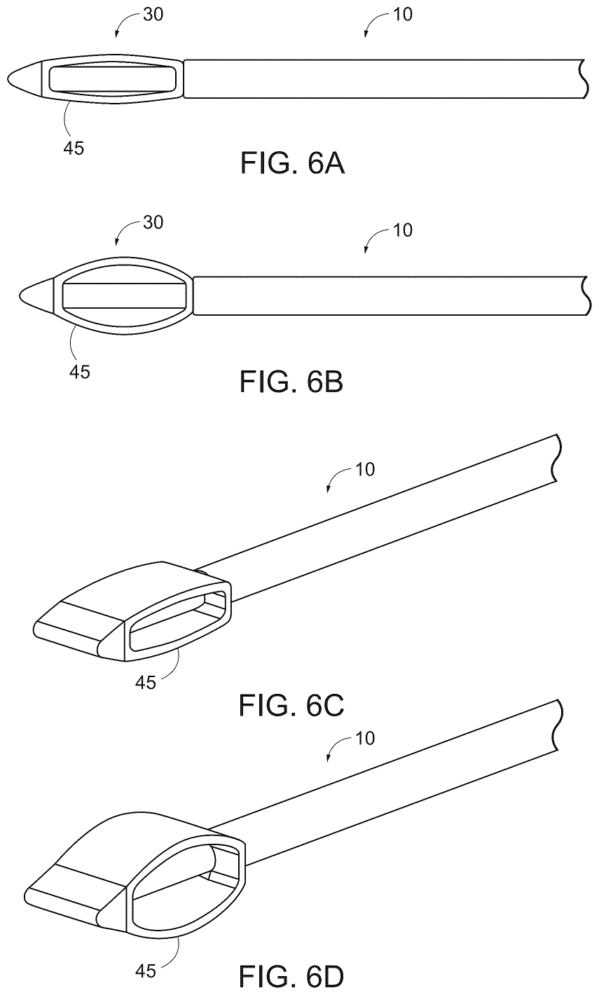

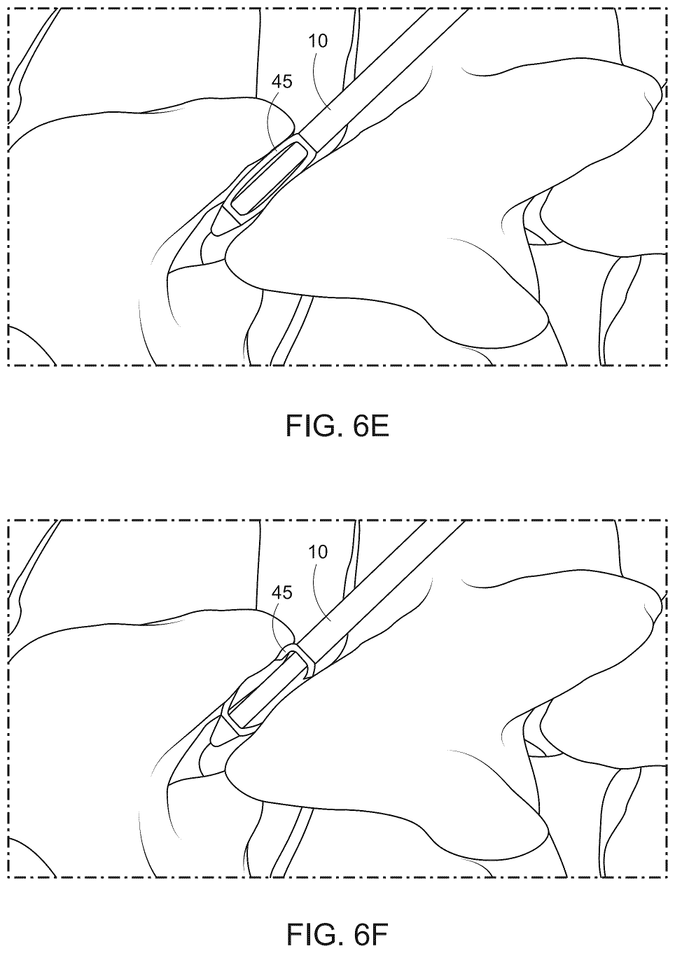

In some aspects, and as can be understood from FIGS. 5A-6F, the access device 10 includes the body 15 which may be an elongated tubular body having a proximal portion 20 and a distal portion 25. The distal portion 25 further includes a chamfered or beveled end feature 30 positioned at an end of the distal portion of the access device. The end feature 30 may be an expandable end feature including teeth or raised ridges 30a to engage the facet joint and/or the stop feature or the raised/protruding features described in FIG. 4.

As shown in FIGS. 5A-5D, in one aspect, the end feature 30 comprises two arms 40 held in a closed position by a detent feature 42 and expanded by actuation of an internal rod 44 when positioned in the facet joint (see FIGS. 5E-5F showing placement in the facet joint). The internal rod may open the arms at a pivot point or the arms may have an internal edge with a ramp feature that is engaged by the rod as the rod is advanced distally.

As illustrated in FIGS. 6A-6D, in another aspect, the end feature comprises an expandable member 45 that is delivered in a flat or closed position (see FIG. 6E) and is expanded or opened once in position in the facet joint (FIG. 6F). The expandable member is opened via an inner rod connected to the tip that is actuated proximally to compress the expandable member. Alternatively, an outer rod actuates distally to compress the expandable member. The expandable member is made of nitinol, stainless steel, spring steel or other metal or metal alloy that can expand and contract without permanent deformation.

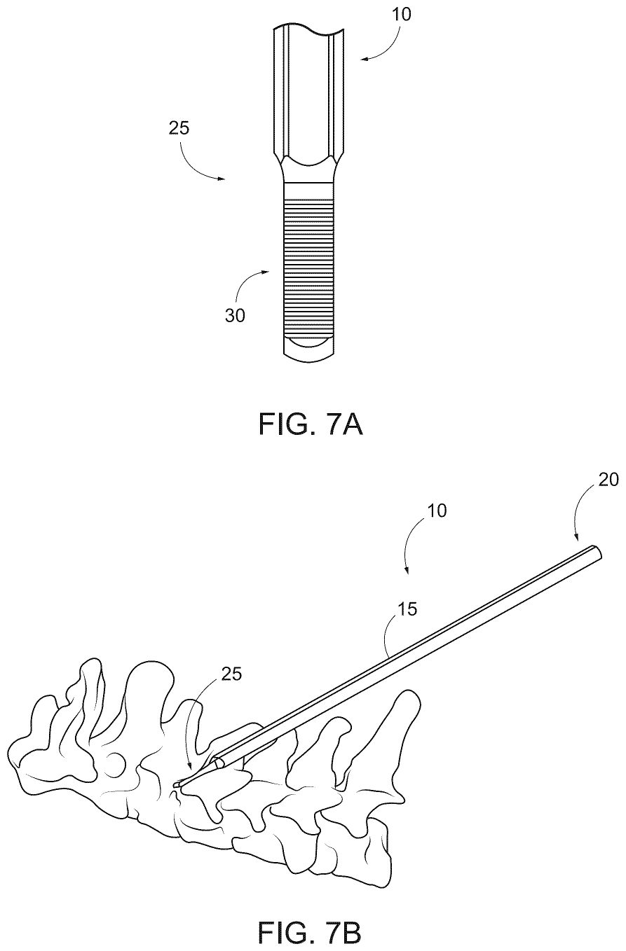

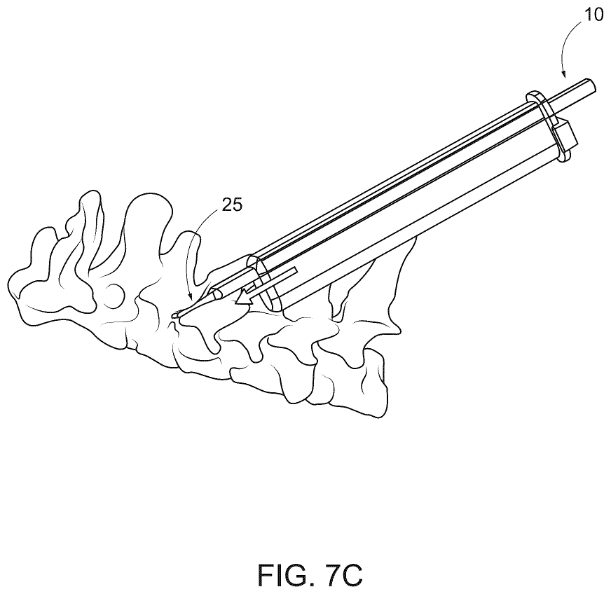

In some aspects, and as can be understood from FIGS. 7A-7C, the access device 10 includes the body 15 which may be an elongated body having a proximal portion 20 and a distal portion 25. The distal portion 25 further includes a chamfered or beveled end feature 30 positioned at an end of the distal portion of the access device. The end feature 30 may be a rigid tip 50 (7A). In use, and as seen in FIGS. 7B-7C, the device 10 is inserted in the facet space and is used in conjunction with other instruments (such as the guide tube shown in FIG. 7C) to guide a screw (depicted by an arrow in FIG. 7C) into the lateral mass at the same angle as the facet joint.

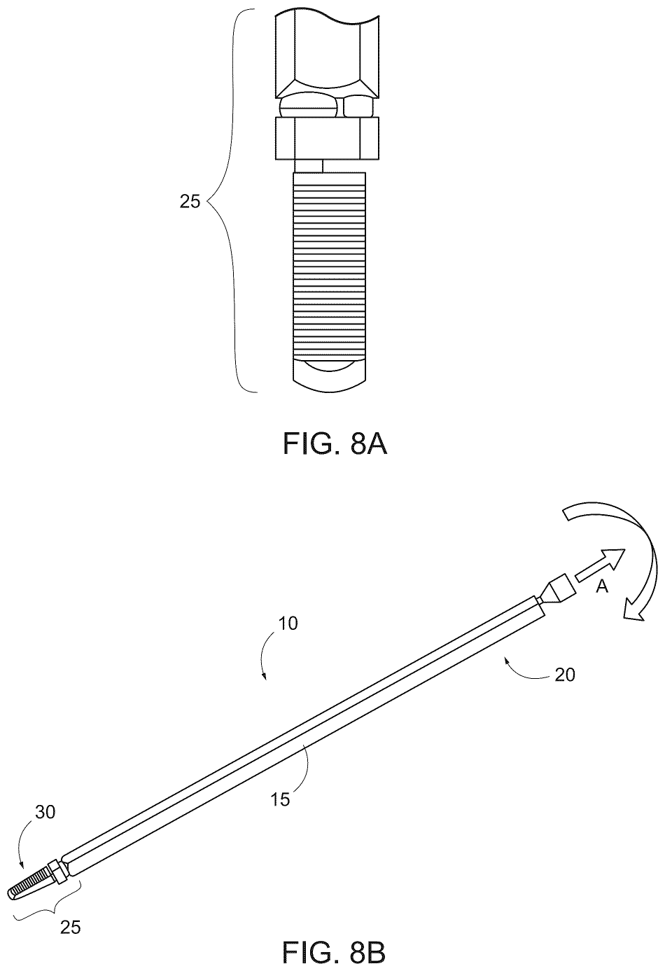

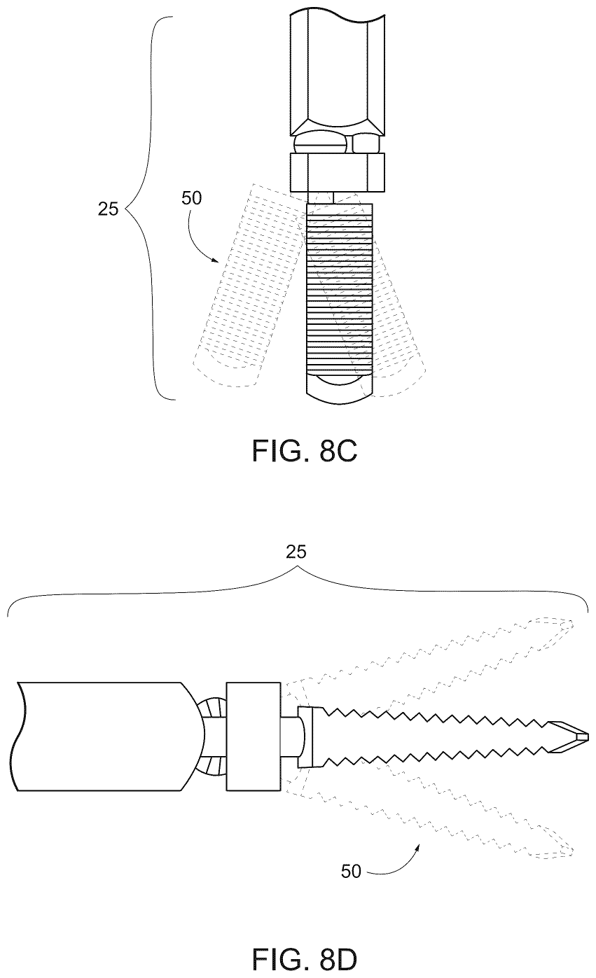

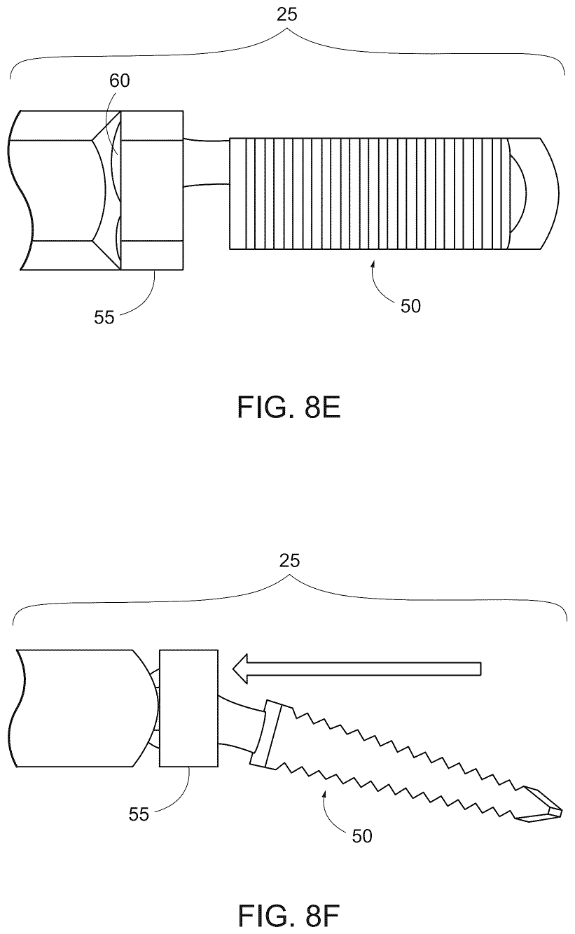

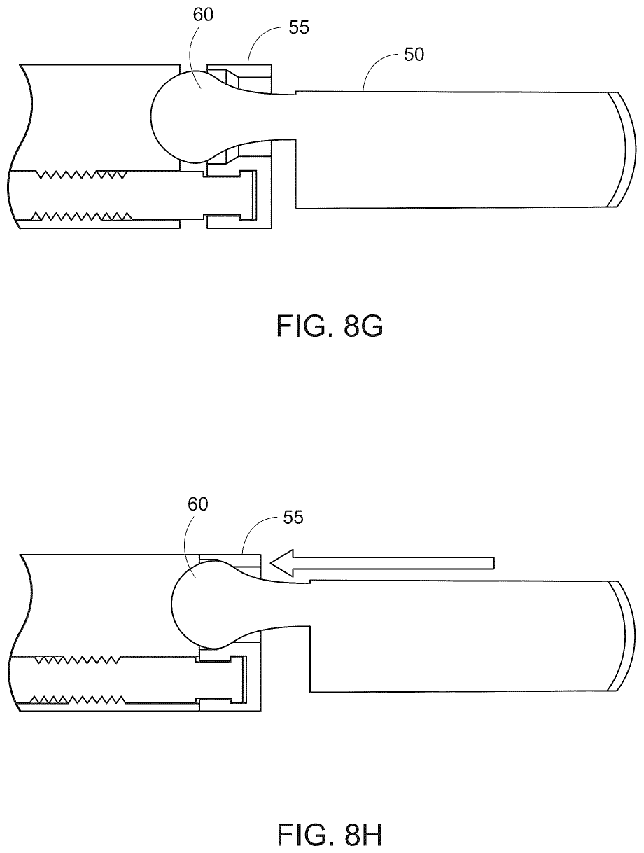

In some aspects, and as can be understood from FIGS. 8A-12H, the access device 10 includes the body 15 which may be an elongated body having a proximal portion 20 and a distal portion 25. The distal portion 25 further includes a chamfered or beveled end feature 30 positioned at an end of the distal portion of the access device. The end feature 30 may include an articulating tip 50.

As indicated in FIG. 8A, the articulating tip 50 may comprise a locking ball joint with plate. As shown in FIG. 8B, the user pulls in the direction of arrow A and rotates the handle clockwise to lock the articulating tip into place. FIGS. 8C-8D show the articulating tip in an open or unlocked position in which the tip is permitted to rotate freely. FIG. 8E-8F show the articulating tip in a locked position. The plate 55 is engaged with the ball joint 60 which locks the tip 50 in place. FIGS. 8G-8H illustrate section views, with FIG. 8G showing the tip 50 in an open position, where the plate 55 is not engaged with a ball joint 60. FIG. 8H shows the plate 55 is locked into place via threads along the shaft of the distal portion of the body 15. The ball joint 60 is engaged with the plate 55.

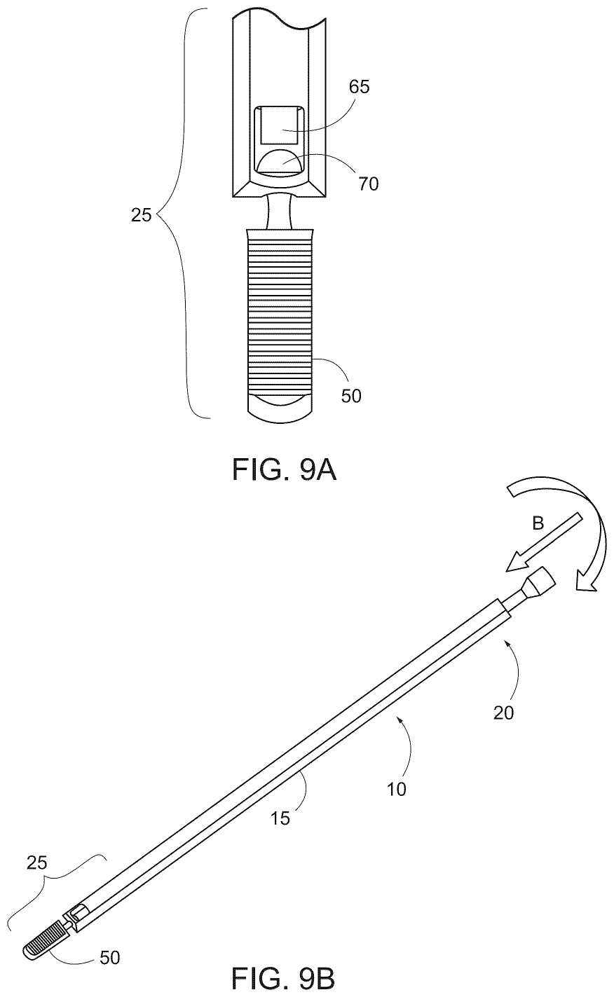

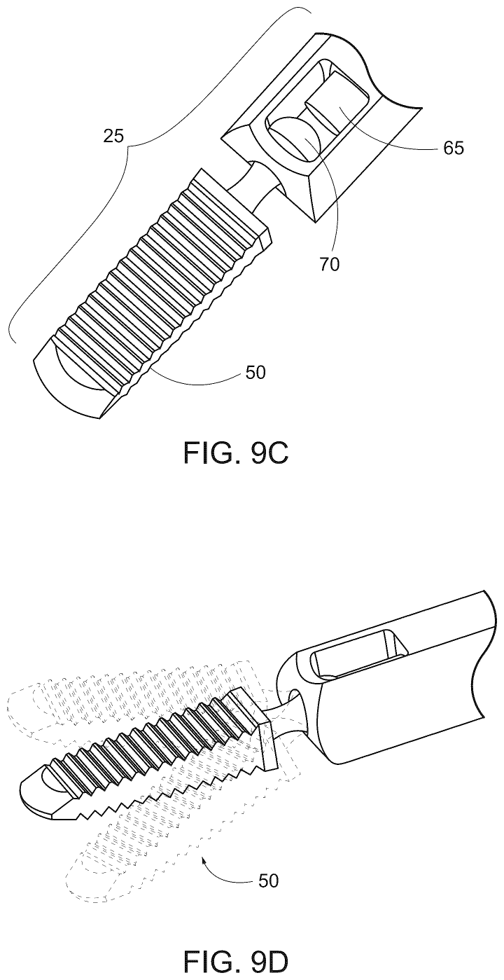

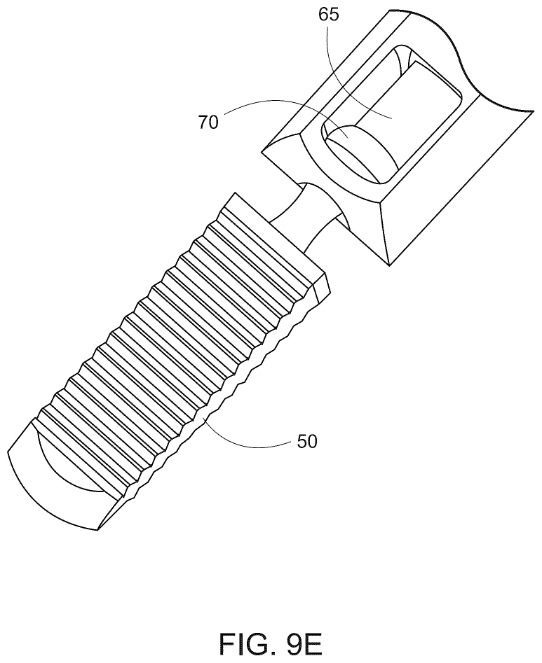

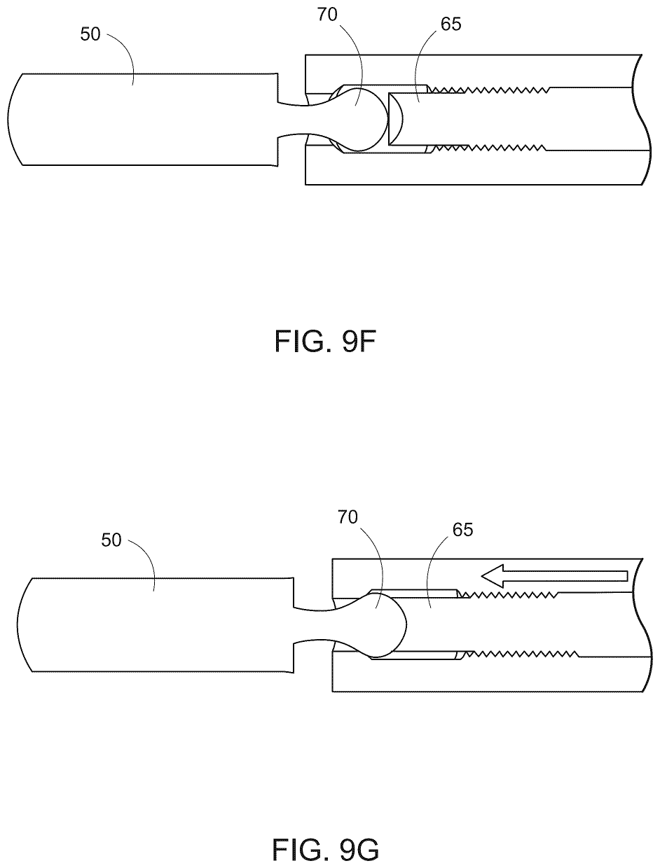

As indicated in FIG. 9A, the articulating tip 50 comprise a locking ball joint with a housing. As shown in FIG. 9B, the user pushes down on the handle in the direction of arrow B and rotates the handle clockwise to lock the articulating tip in place. FIGS. 9C-9D show the articulating tip 50 in an open or unlocked position in which the tip is permitted to rotate freely. FIG. 9E shows the tip 50 in a locked position. As shown, a rod 65 is engaged with a ball joint 70, thereby locking the tip 50 in place. FIGS. 9F-9G illustrate section views, with FIG. 9F showing the tip 50 in an unlocked position, where the rod 65 is disengaged with the ball joint 70 and tip 50. FIG. 9G shows the tip 50 in a locked position where the rod 65 is engaged with the ball joint 70 and tip 50.

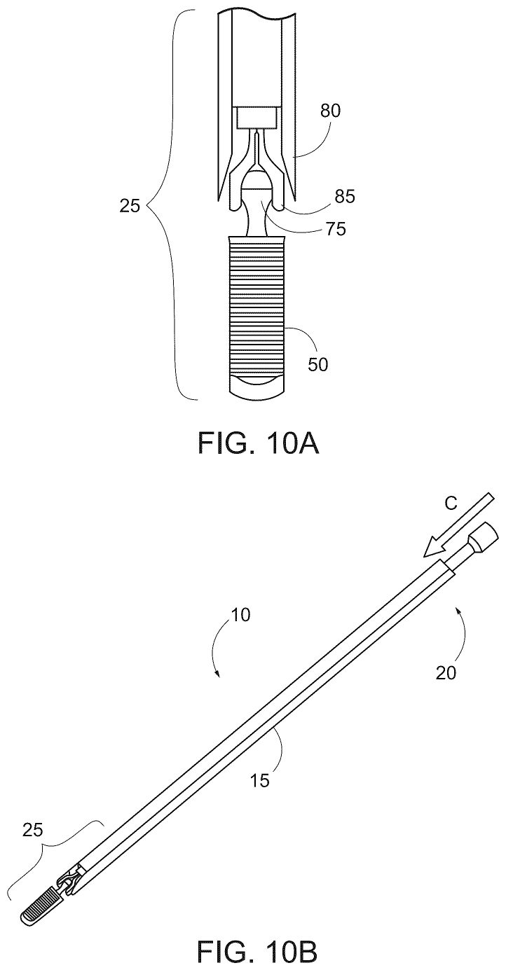

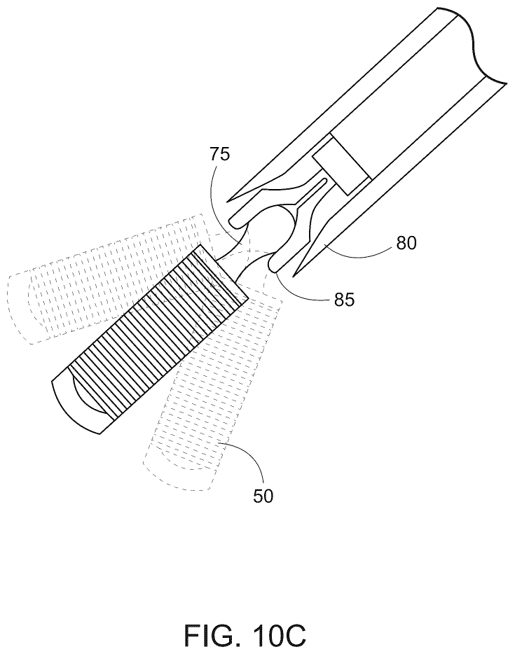

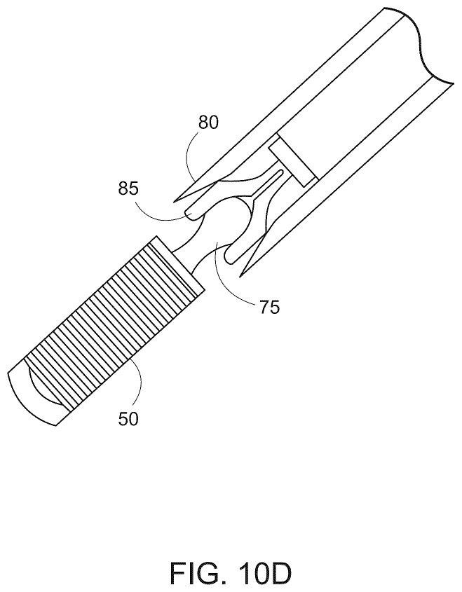

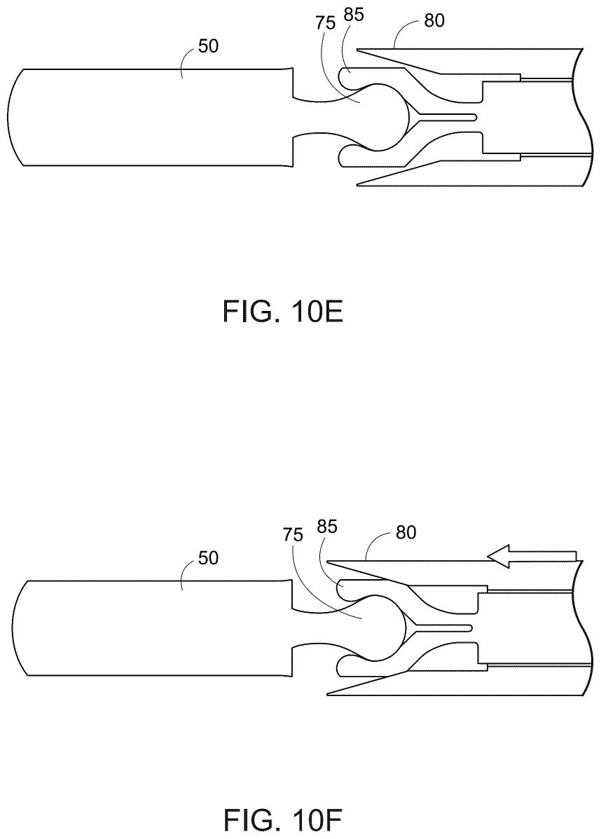

As indicated in FIG. 10A, the articulating tip 50 comprises a sleeve 80 with a tulip 85. As shown in FIG. 10B, the user presses down on the handle in the direction of arrow C to lock the articulating tip 50 in place. FIG. 10C show the articulating tip 50 in an open or unlocked position in which the tip is permitted to rotate freely. FIG. 10D shows the tip in a locked position. As shown, a sleeve 80 and tulip 85 are engaged with a ball joint 75 thereby locking the tip 50 in place. FIGS. 10E-10F illustrate section views, with FIG. 10E showing the tip 50 in an unlocked or open configuration. The sleeve is disengaged with the locking tulip and the tip 50 is able to freely rotate. FIG. 10F shows the tip in a locked position. The sleeve 80 is engaged with the locking tulip 85 thereby locking the ball joint 75 and thereby the tip 50 in place.

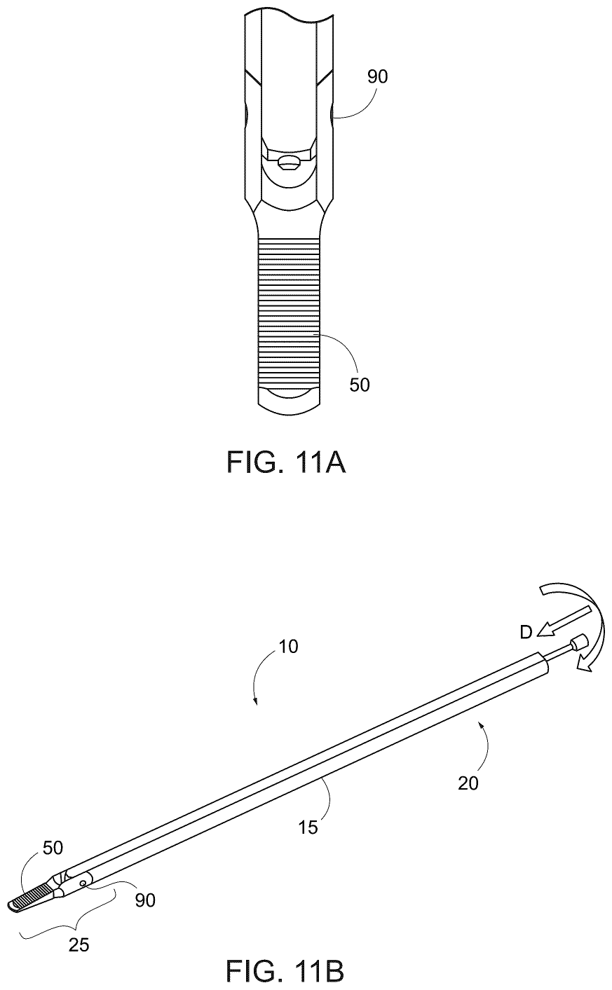

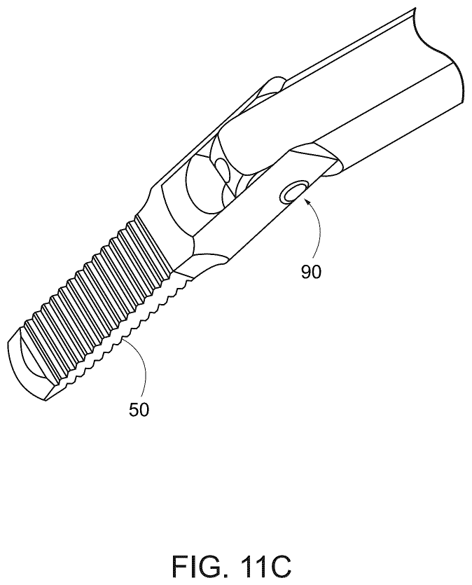

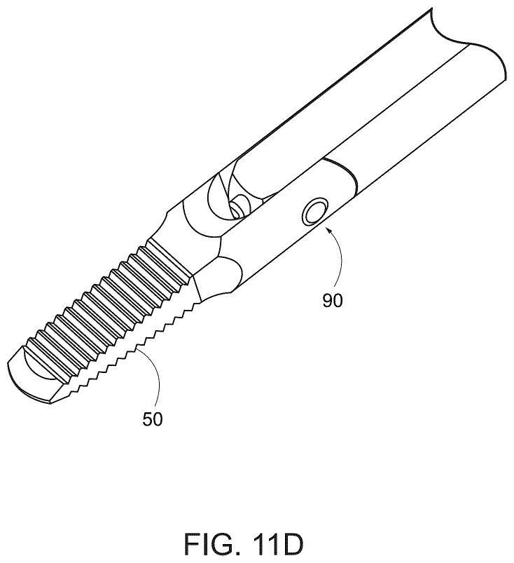

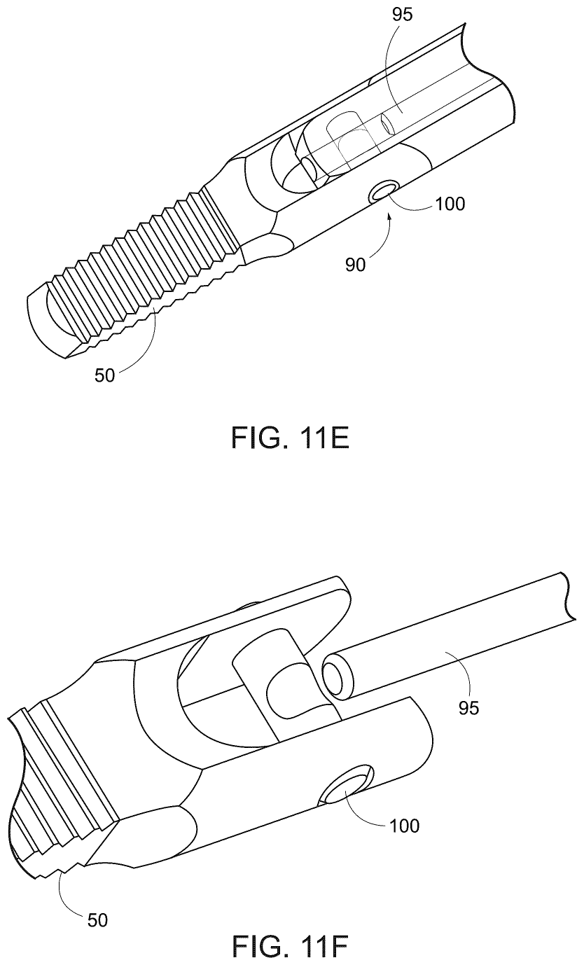

As indicated in FIG. 11A, the articulating tip 50 comprises a single direction pivot 90. As shown in FIG. 11B, the user pushes down on the handle in the direction of arrow D, and turns the handle clockwise to lock the tip 50 in place. FIG. 11C illustrates the tip 50 in an open or unlocked position. FIG. 11D illustrates the tip 50 in a closed or locked position. FIGS. 11E-F are transparent and enlarged views showing the tip 50 in an unlocked position. A rod 95 is not engaged with a locking dowel pin 100 and the tip 50 is able to rotate along the coronal plane. FIGS. 11G-H are transparent and enlarged views showing the tip 50 in a locked position. The rod 95 is pushed forward to interact with the locking dowel pin 100 thereby locking the tip 50 in one position in the coronal plane.

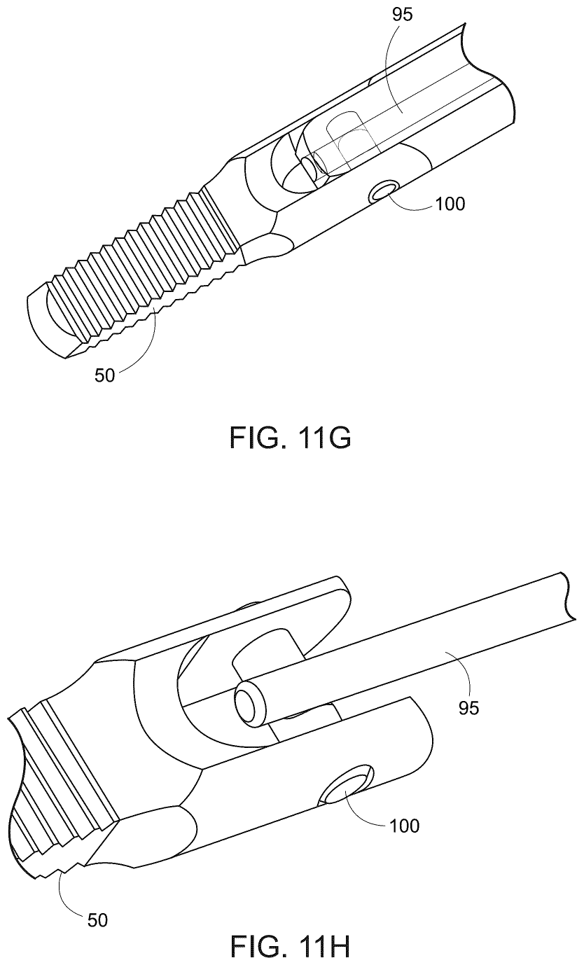

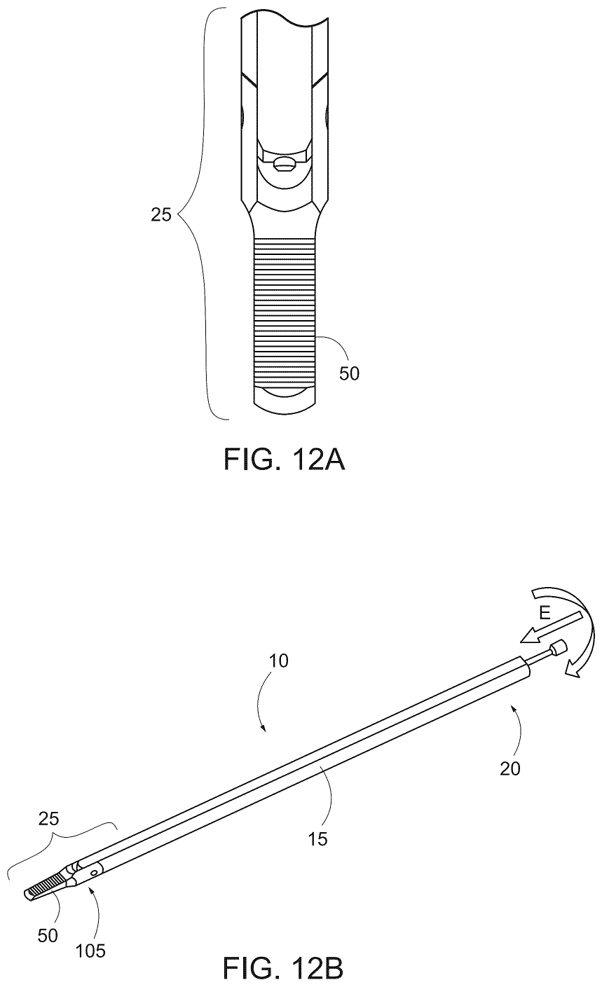

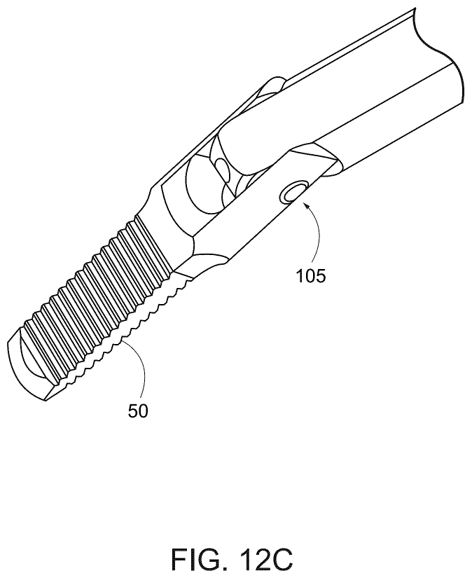

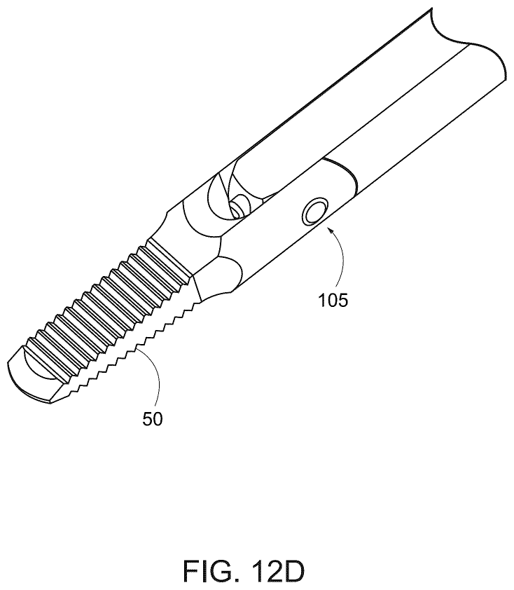

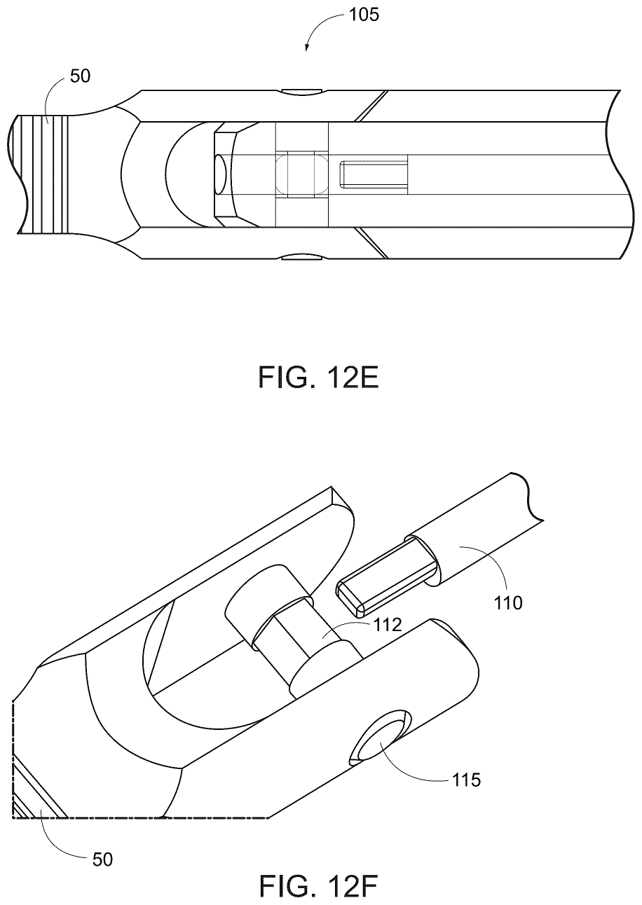

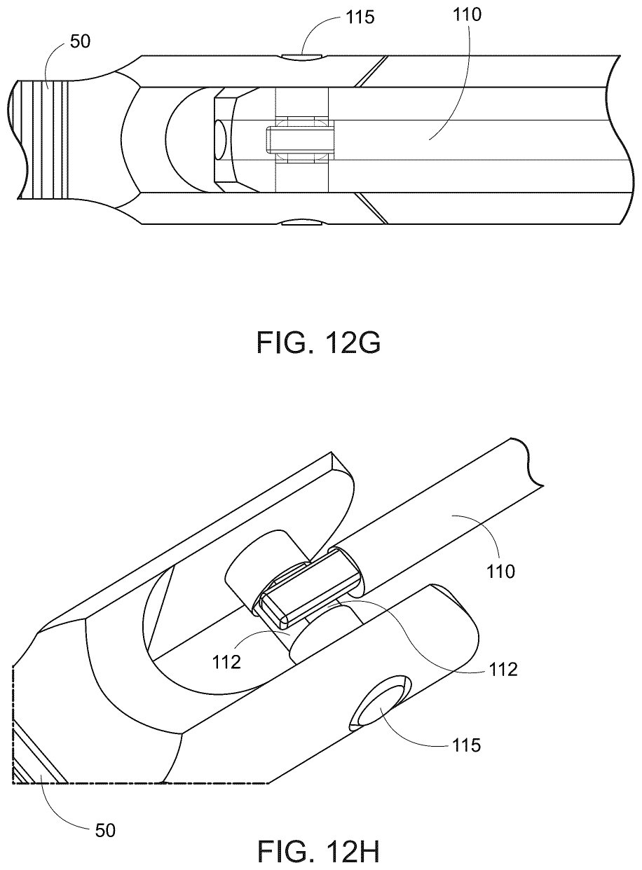

As indicated in FIG. 12A, in some aspects, the articulating tip 50 comprises a locking wheel 105. As shown in FIG. 12B, the user pushes down on the handle in the direction of arrow E and turns the handle clockwise to lock the tip in place. FIG. 12C illustrates the tip 50 in an open or unlocked position. FIG. 12D illustrates the tip 50 in a closed or locked position. FIGS. 12E-F are transparent and exploded views showing the tip in an unlocked position. A rod 110 is disengaged from a locking feature 112 on a dowel pin 115 thereby allowing the tip 50 to rotate freely. FIGS. 12G-H are transparent and exploded view showing the tip 50 in a locked position. The rod 110 is pushed forward to interact with the flat locking feature 112 of the dowel pin 115 thereby locking the tip 50 into place. Depending on which portion of the flat locking feature is engaged, the tip 50 can be locked in one of six different positions in the coronal plane. In other embodiments, the flat locking feature may have more than six positions or less than six positions.

As noted above, the access device may be used with other tools to deliver a spinal fixation device. The delivery may be achieved with, for example, a guide portal or device.

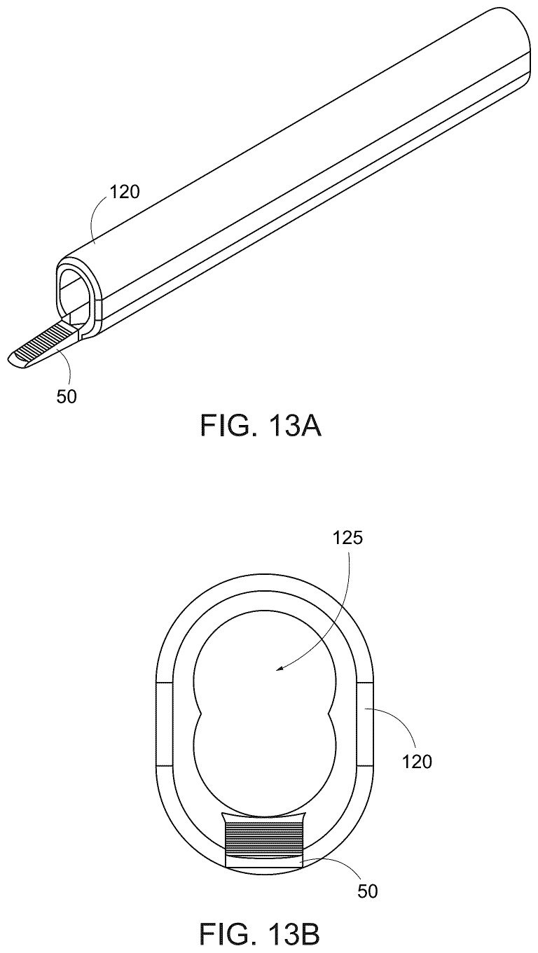

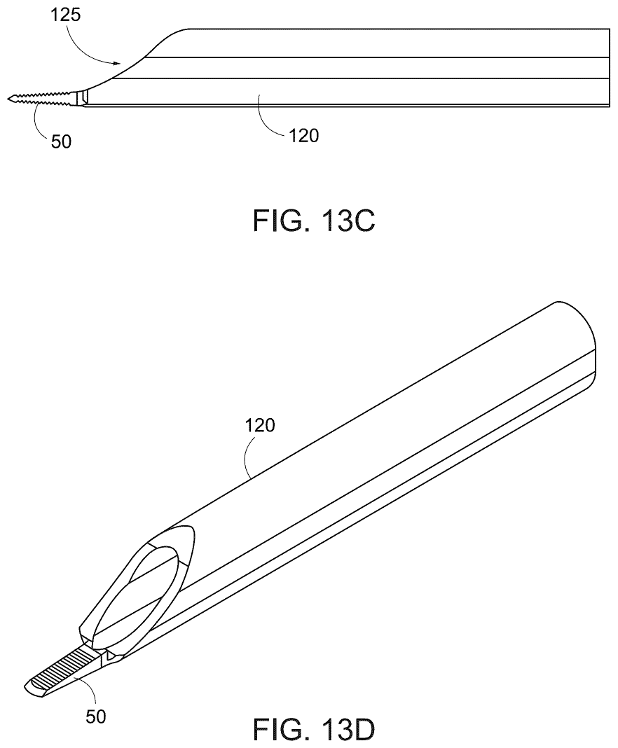

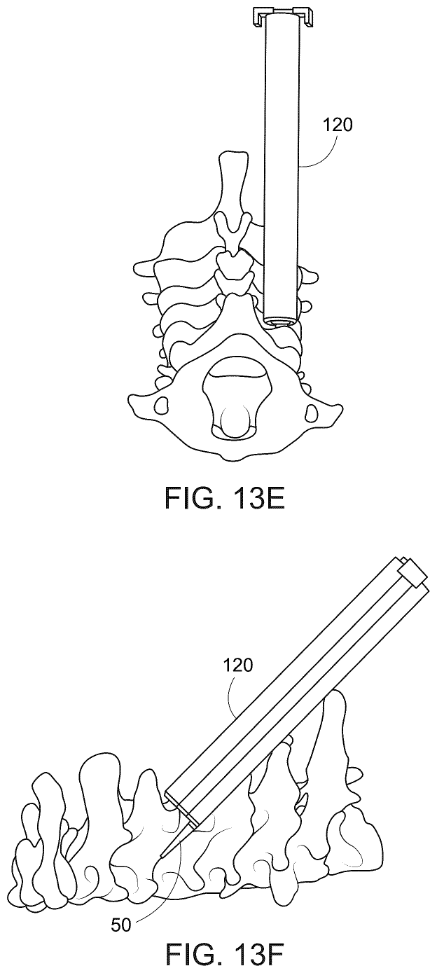

As shown in FIGS. 13A-13F, in some aspects, the access device 10 or the tip 50 is combined with a guide portal as a single, integral or monolithic access and guide system. As indicated in FIGS. 13A-B, the guide portal 120 comprises an elongated tubular body defining at least one lumen 125 therethrough. In some aspects, there may be more than one lumen or there may be partial lumen. FIGS. 13C-D illustrate another embodiment of the access and guide system the guide portal 120 comprises an elongated tubular body defining at least one lumen 125 therethrough. The tip 50 and the facet engaging face of the guide portal 120 are tapered to aid in insertion and access to the facet space. In use, and as shown in FIGS. 13E-F, the access and guide system is inserted into the facet space. The tip 50 imbeds and fixates the portal 120 to the angle of the facet. After insertion, other instruments in the system, such as a spinal fixation device, pass through the portal to the lateral mass at the same angle as the facet. While FIGS. 13E-F show insertion of the embodiment shown in FIGS. 13A-B, it is understood that the embodiment of FIGS. 13C-D may be similarly inserted.

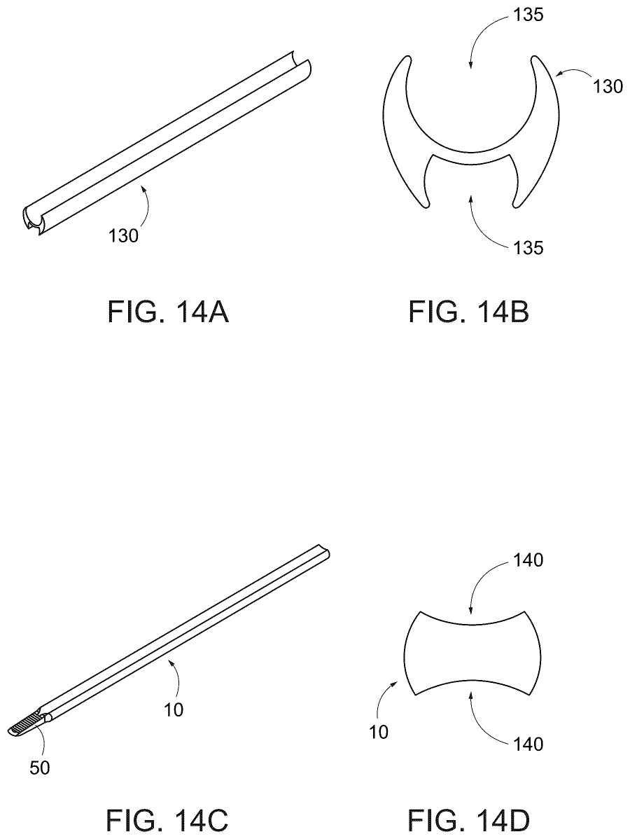

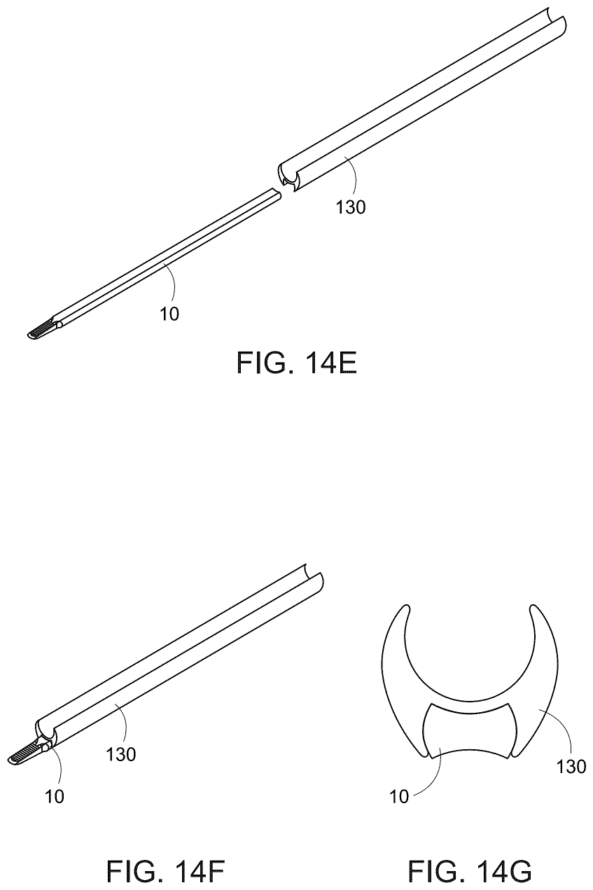

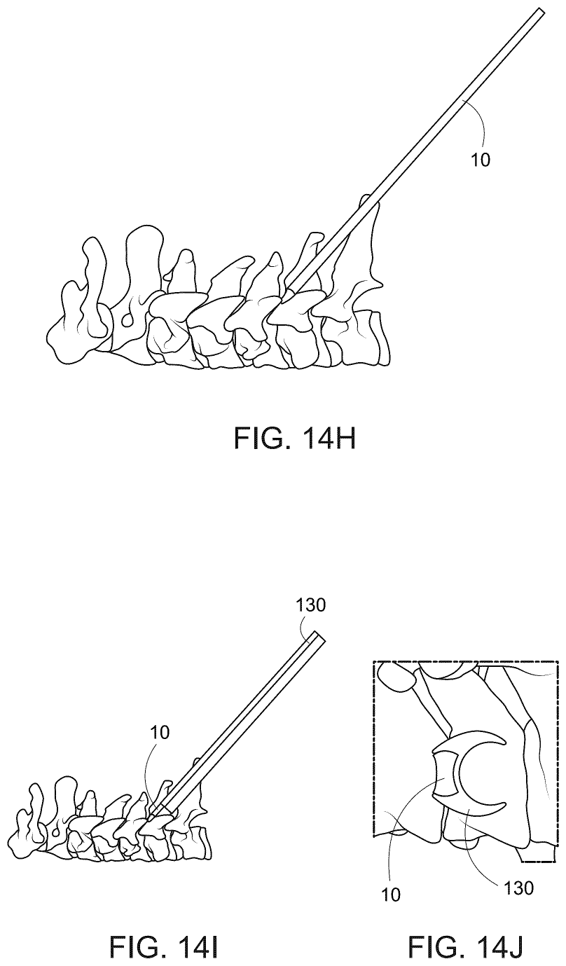

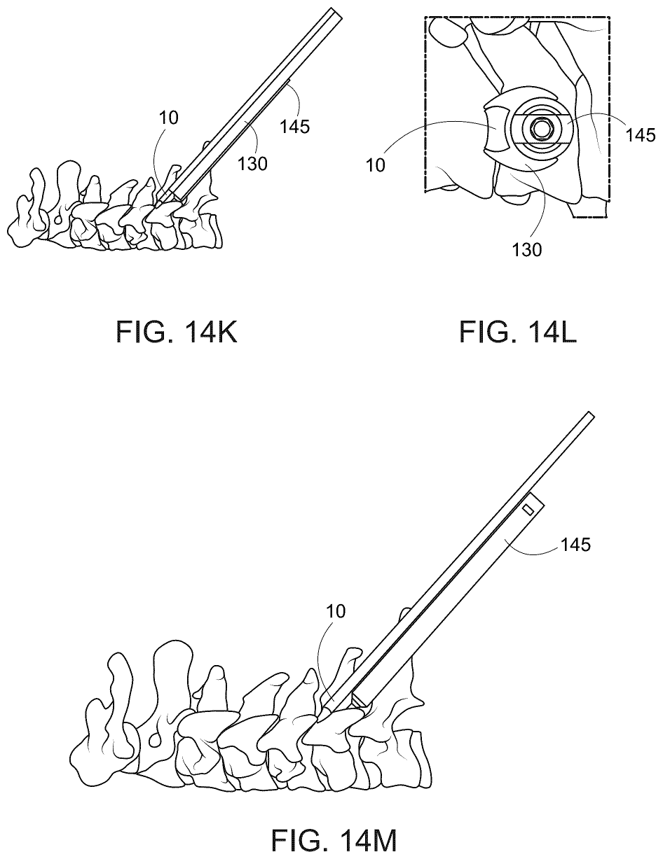

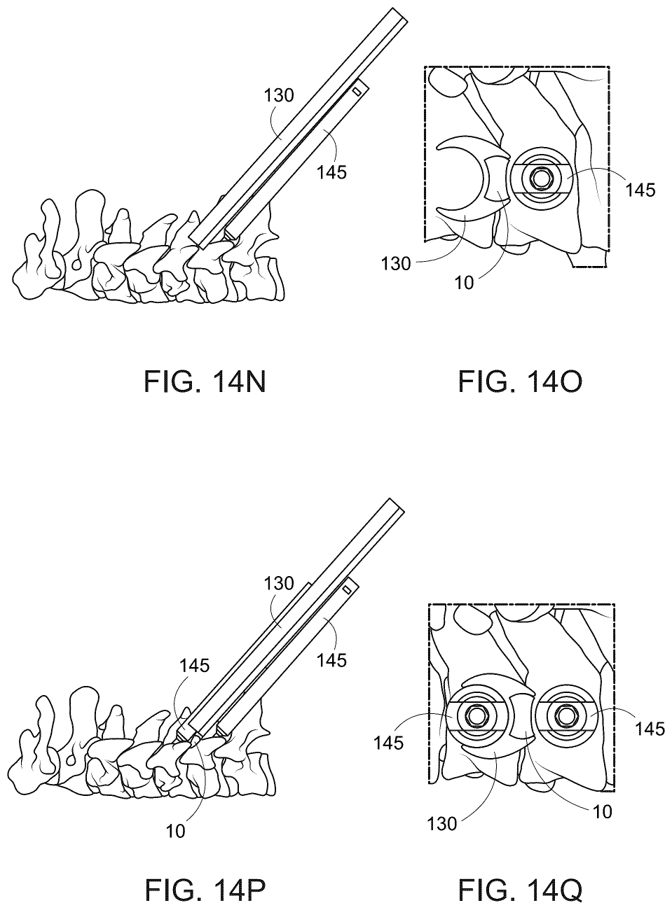

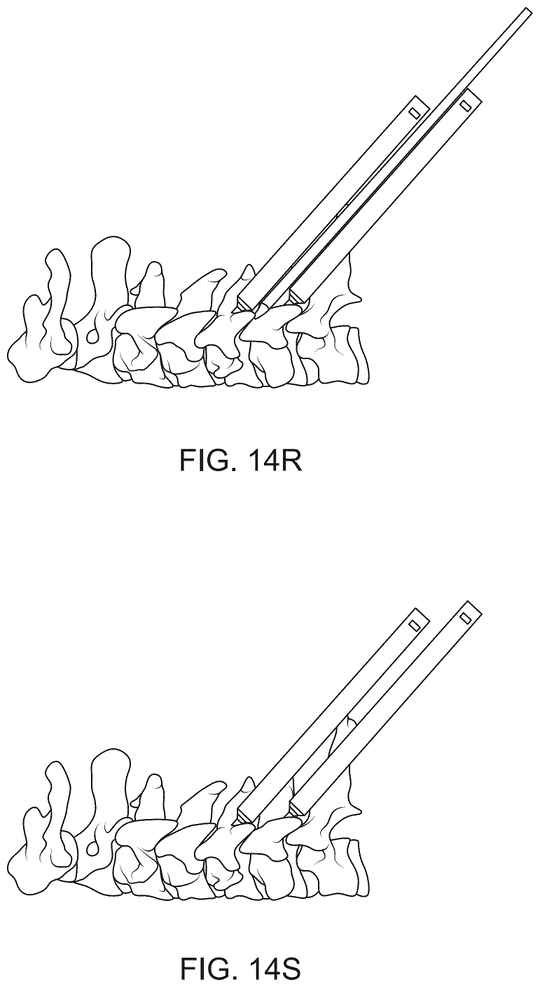

As illustrated in FIGS. 14A-19Q, in some embodiments, an access device 10 is keyed or paired to a guide device 130 as components of an access and delivery system. The guide device may be used to guide a spinal fixation device to the spine.

As shown in the perspective and cross section views of FIGS. 14A-D, in one embodiment, the guide device 130 includes a plurality of slots or openings 135, an upper or first slot configured to receive and guide a tower or screw component of the spinal fixation device (see more description below) and a lower or second slot configured to engage the access device 10. The access device 10 includes first (or upper) and second (or lower) notches or recesses 140 complementary to and keyed to the guide device. FIGS. 14E-G illustrate the access device 10 and guide device 130 positioned together and FIGS. 14H-J illustrate the access device 10 inserted into the facet joint and then the guide device inserted over the guide device. FIGS. 14K-14S depicts use of the access device 10 and guide device 130 to deliver one or more spinal fixation devices 145. In use, the access device 10 inserted into the facet joint and then the guide device inserted over the access device. The spinal fixation device 145 is inserted through the guide 130 and the bone screw is screwed into the lateral mass (FIGS. 14K-L). The guide 130 is removed (FIG. 14M) but the access device 10 remains in place. The guide is then placed on the other side of the access device to engage the other lateral mass. (FIGS. 14N-O). The spinal fixation device 145 is inserted through the guide 130 and the bone screw is screwed into the lateral mass. (FIGS. 14P-Q). The guide device and the access device are then removed (FIGS. 14R-S) and a rod may be inserted in the towers of the fixation devices (not shown, but see below).

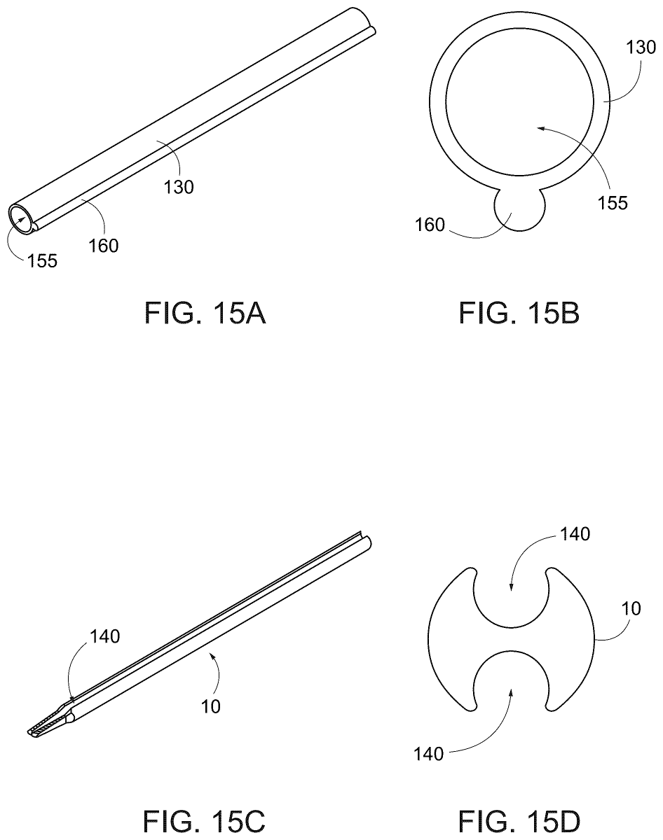

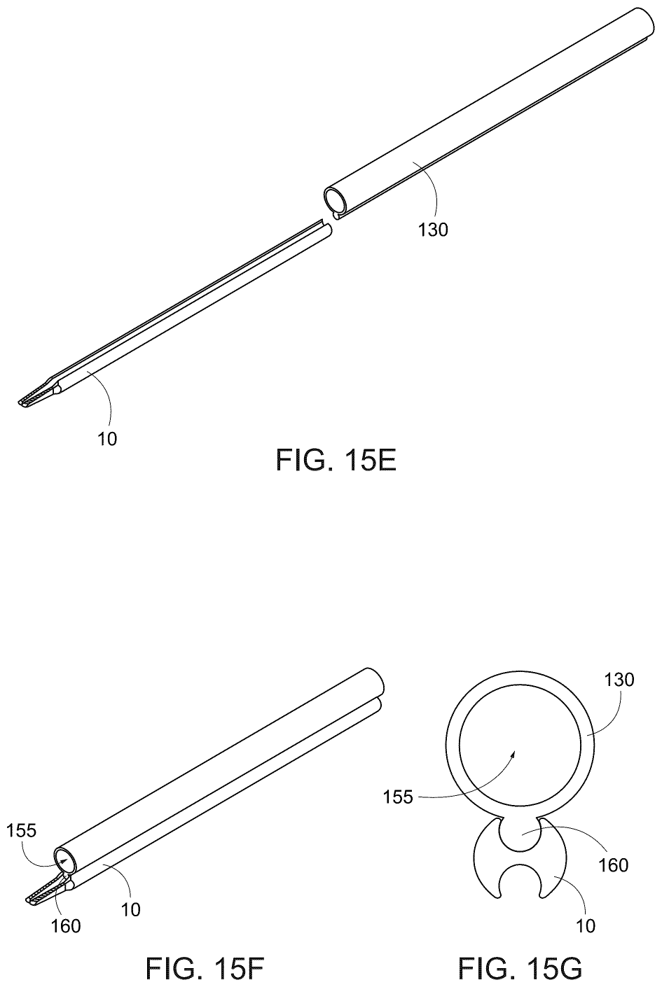

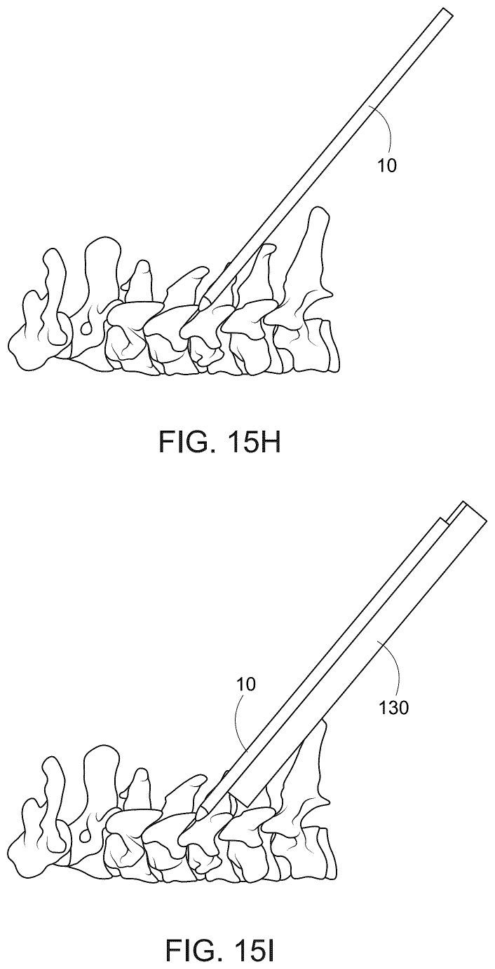

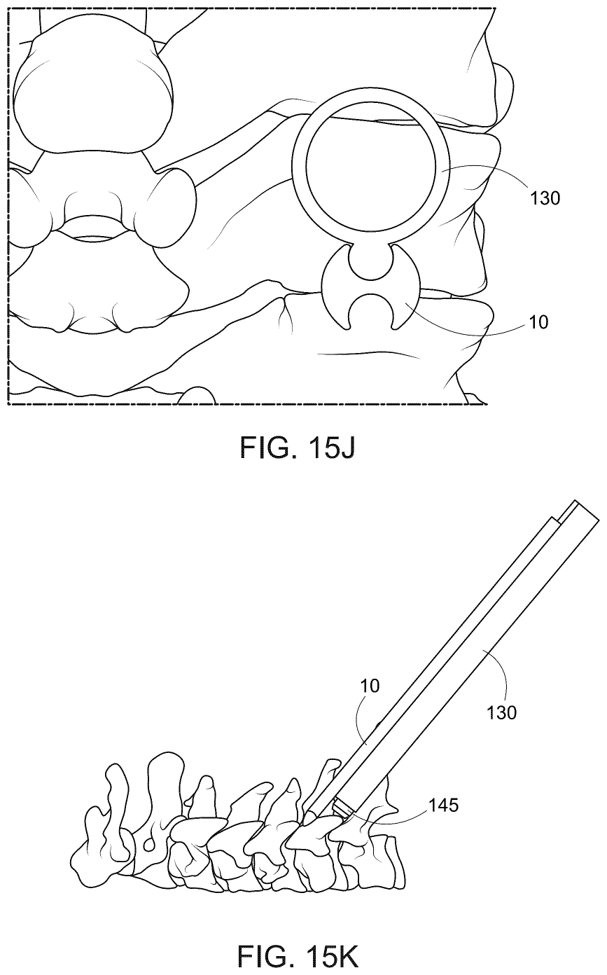

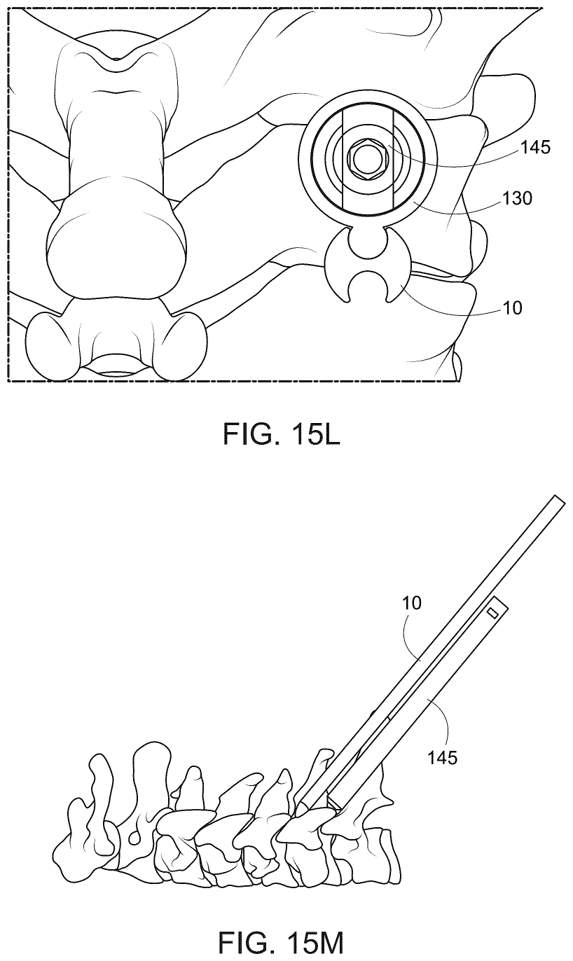

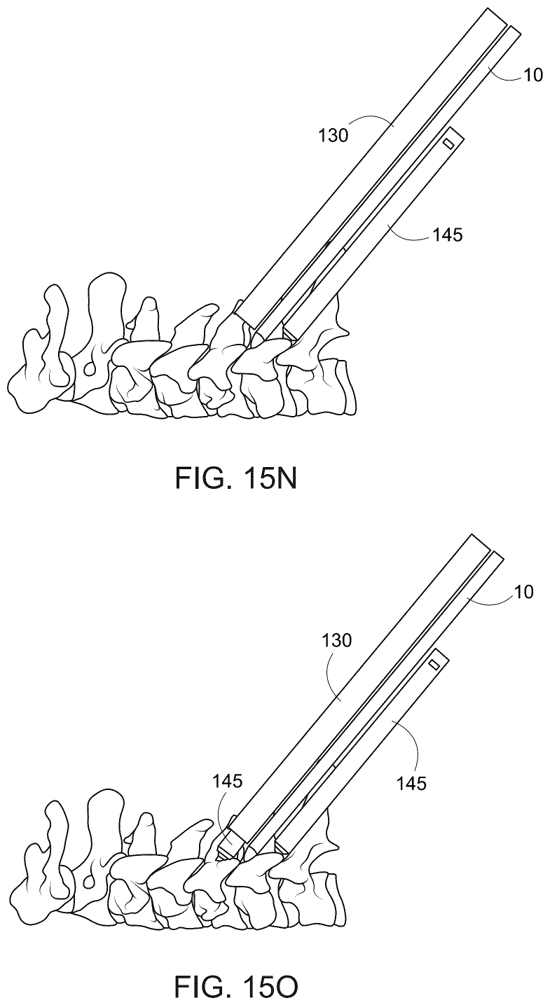

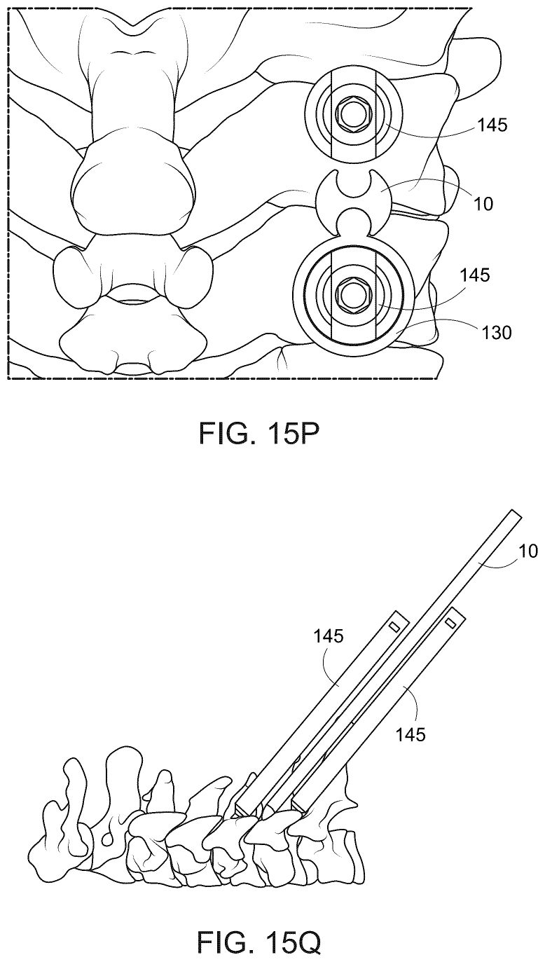

As shown in the perspective and cross section views of FIGS. 15A-D, in one embodiment, the guide device 130 includes a lumen 155 defined therethrough and configured to receive and guide a tower or screw component of the spinal fixation device (see more description below) and a rail mating feature 160 configured to engage the access device 10. The access device 10 includes first (or upper) and second (or lower) notches or recesses 140 complementary to and keyed to the guide device. FIGS. 15E-G illustrate the access device 10 and guide device 130 positioned together and FIGS. 15H-J illustrate the access device 10 inserted into the facet joint and then the guide device inserted alongside the guide device. FIGS. 15K-15R depicts use of the access device 10 and guide device 130 to deliver one or more spinal fixation devices 145. In use, the access device 10 inserted into the facet joint and then the guide device inserted alongside the guide device. The spinal fixation device 145 is inserted through the guide 130 and the bone screw is screwed into the lateral mass (FIGS. 15K-L). The guide 130 is removed (FIG. 15M) but the access device 10 remains in place. The guide is then placed on the other side of the access device to engage the other lateral mass. (FIGS. 15N-O). The spinal fixation device 145 is inserted through the guide 130 and the bone screw is screwed into the lateral mass. (FIGS. 15P-Q). The guide device and the access device are then removed (FIG. 15R) and a rod may be inserted in the towers of the fixation devices (not shown, but see below).

In some aspects, guide device 130 may be used with only a portion of the access device 10, such as the tip 50 and may be further stabilized by engagement with a fixation device, such as a tower or a polyaxial screw with a tower feature, that is already implanted.

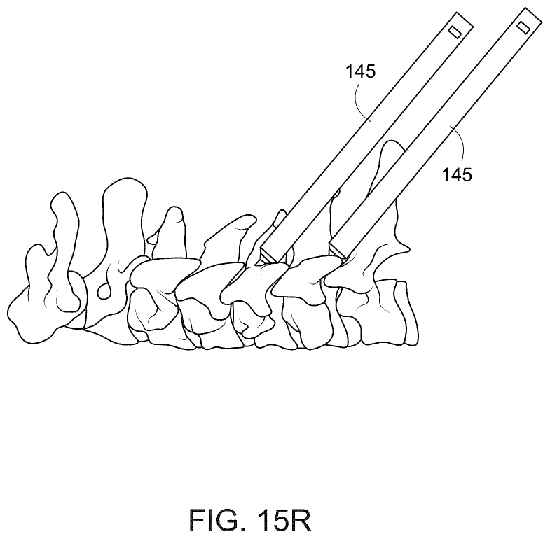

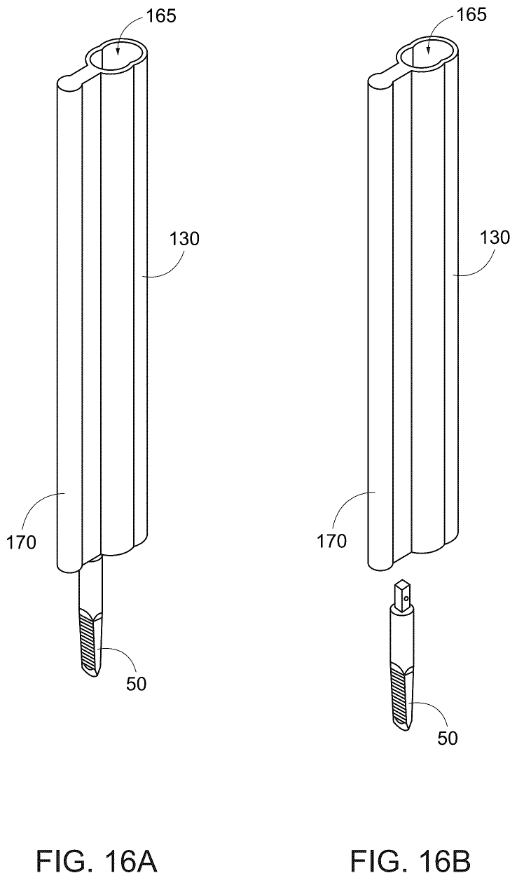

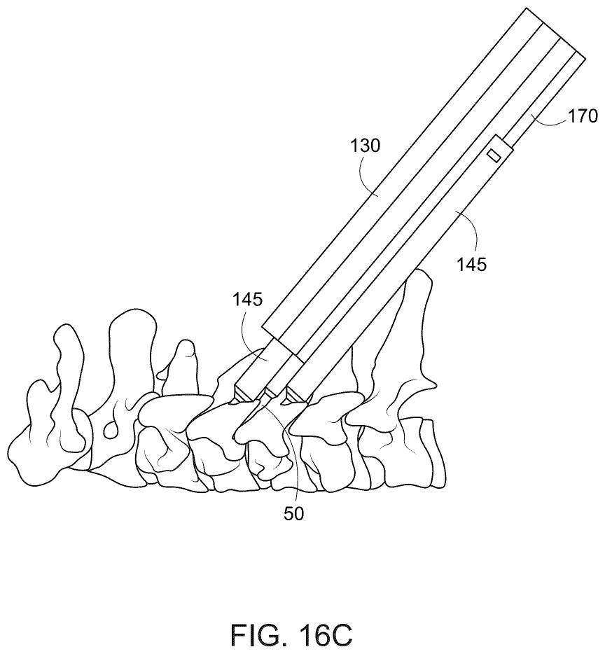

For example, and as shown in FIGS. 16A-B, guide device 130 includes a lumen 165 defined therethrough and a screw tower engagement feature 170. The engagement feature may be a generally cylindrical body protruding from an outer surface of the guide device and generally extending the length of the guide device. The lumen 165 may have the shape of a number eight or other dual positioning shape. FIG. 16C illustrates use of the guide device 130 and access device tip 50 to place towers of the fixation devices, as described in more detail below.

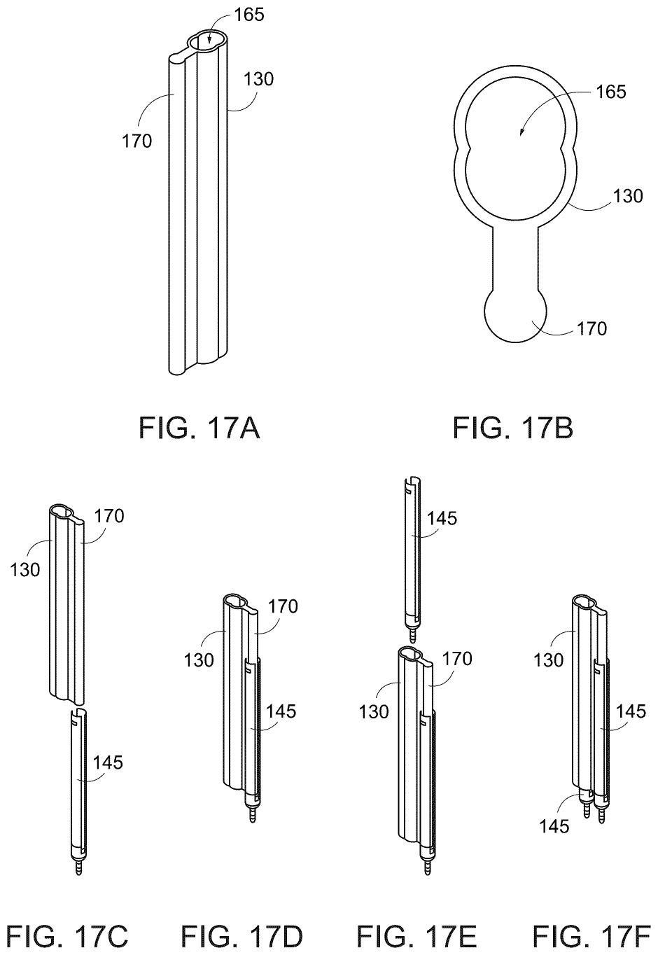

FIGS. 17A-17F illustrate another embodiment of the guide device 130 that may be further stabilized by engagement with a fixation device, such as a tower, that is already implanted. As shown in FIGS. 17A-B, guide device 130 includes a lumen 165 defined therethrough and a tower engagement feature 170. The engagement feature may be a generally cylindrical body protruding from an outer surface of the guide device and generally extending the length of the guide device. The lumen 165 may have the shape of a number eight or other dual positioning shape (indicated by positions 1 and 2). In use (spine is hidden for clarity), and as shown in FIGS. 17C-17F, the guide device 130 is inserted into an anchored tower 145. Then, the second tower 145 is inserted through the lumen 165 into either position 1 or position 2.

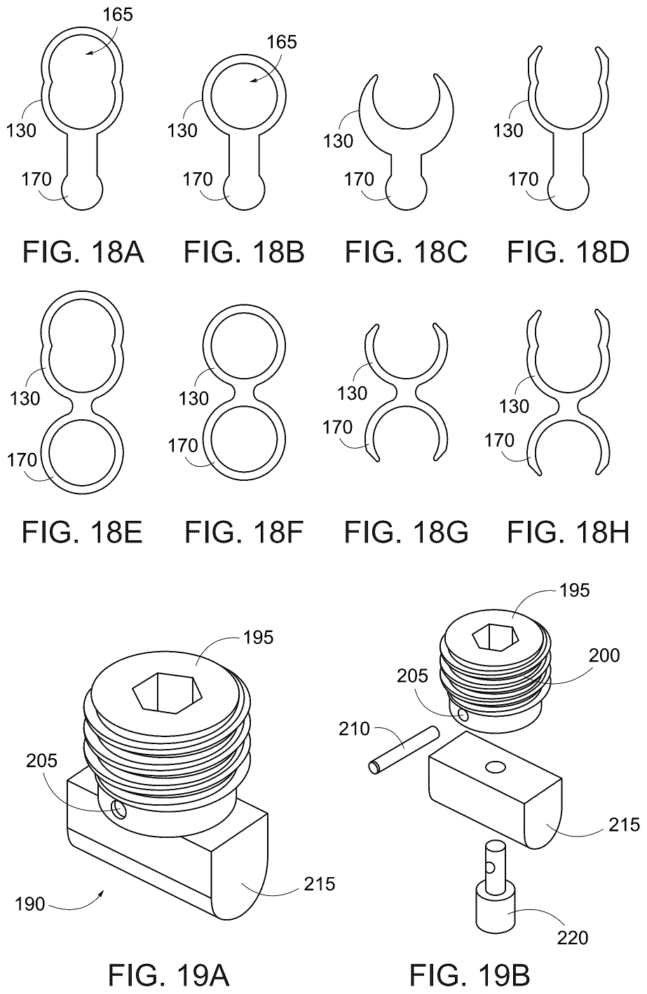

FIGS. 18A-H illustrate cross sections of other embodiments of the guide device 130 including a lumen 165 defined therethrough and a tower engagement feature 170. The tower engagement feature may be configured to be received by the tower (FIGS. 18A-D) or may be configured to receive the tower (FIGS. 18E-18H).

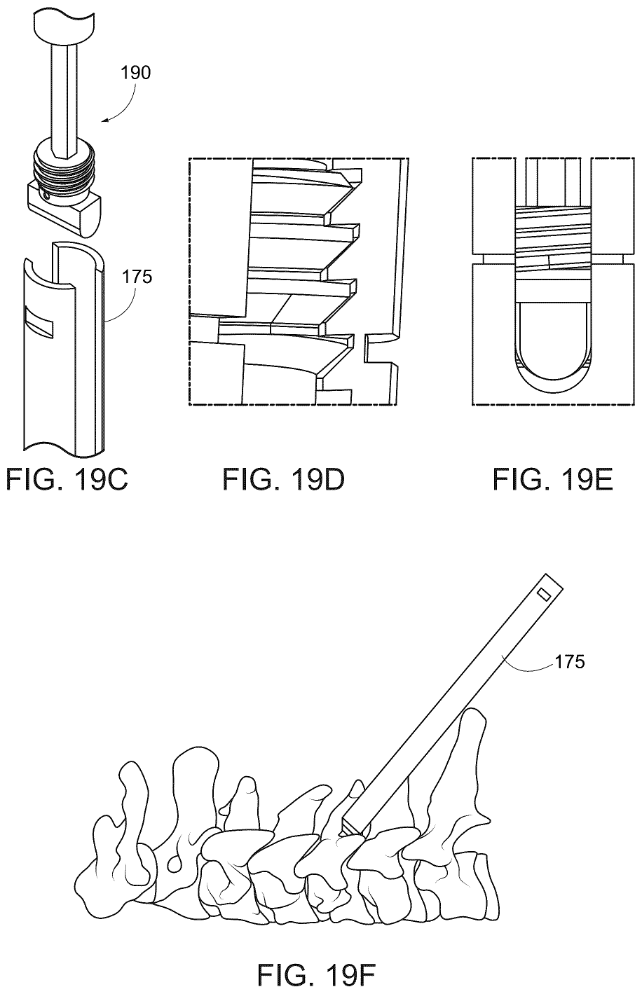

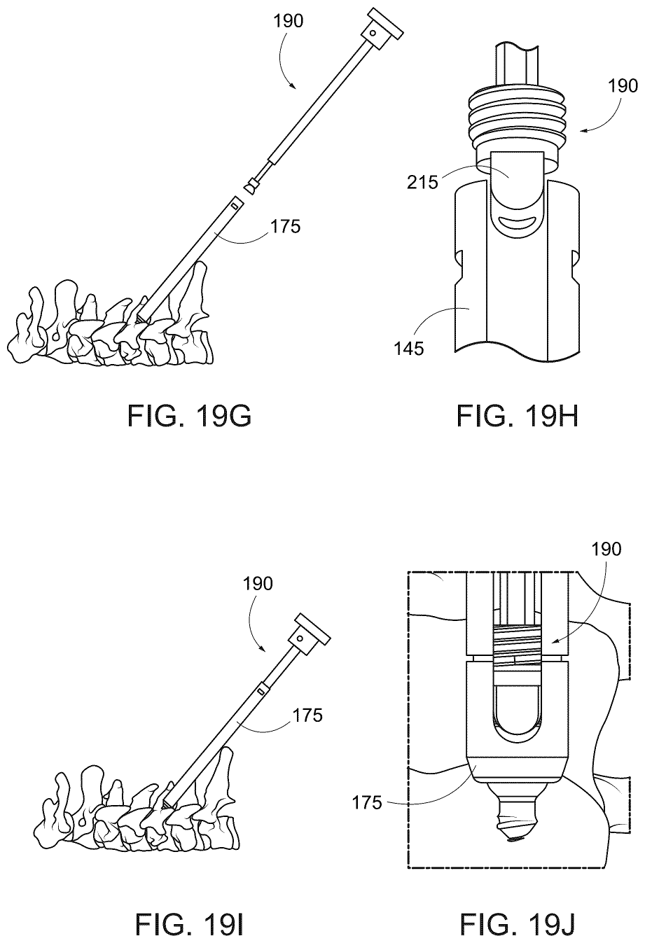

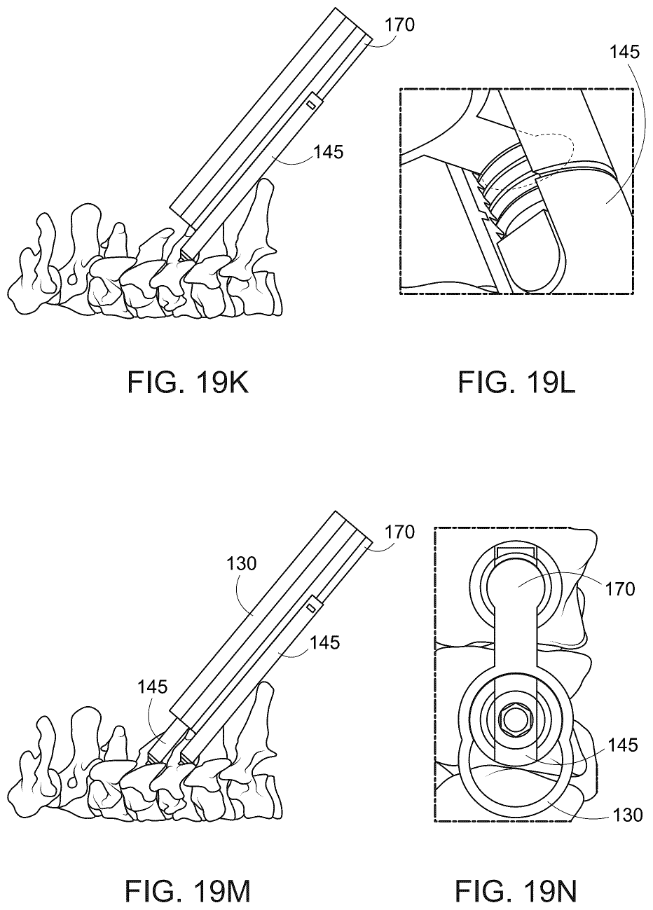

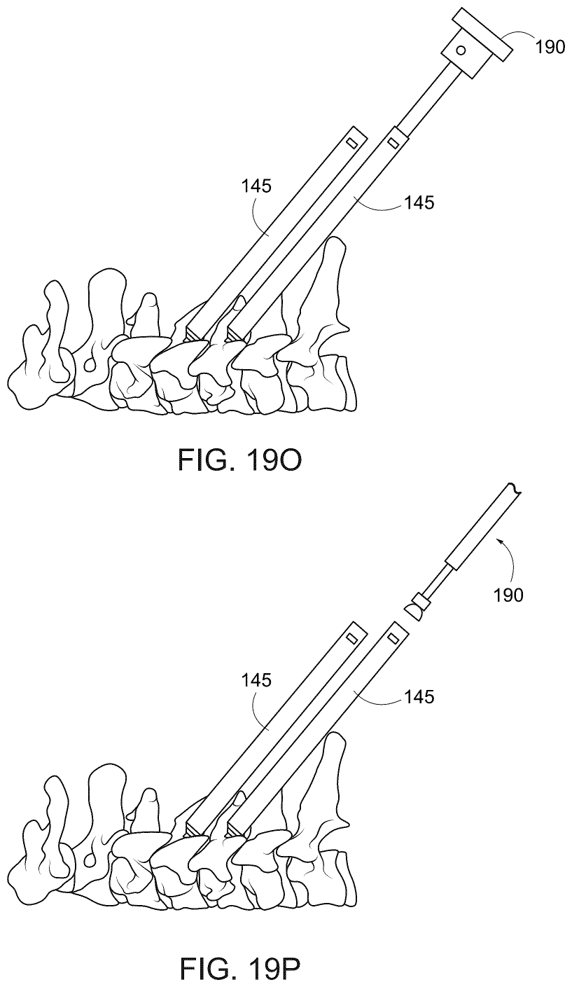

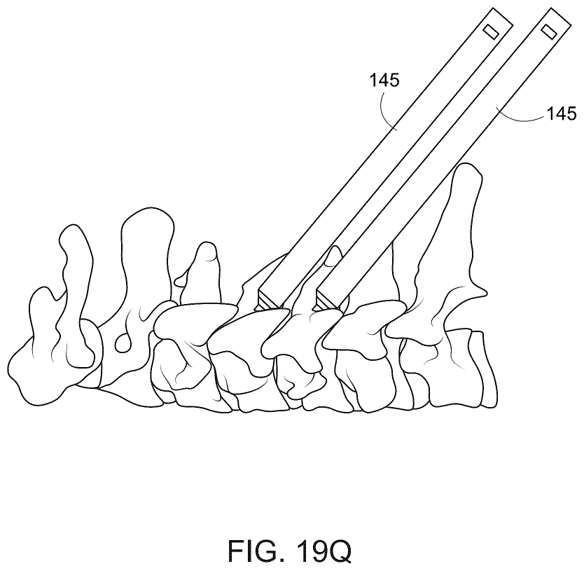

FIGS. 19A-19B depict a temporary locking screw that may be used with the system of FIG. 17. The locking screw 190 includes a hex head 195, external threads 200 that match internal tower threading, a securing pin hole 205, a securing pin 210, a rotating head 215, which may be 3.5 mm, and a pin 220 that allows the head to rotate about the shaft. In use, and as illustrated in FIGS. 19C-19E, the screw 190 is inserted into the tower (19C), and the threading 200 engages with the internal tower threading (19D). The screw head 195 abuts the saddle to lock rotation of the tower with respect to the screw (19E). The tower will not rotate if the screw remains tightened.

In use in the spine, the tower is screwed into the lateral mass (19F). The screw 190 is inserted into the tower 145 with a hex driver (19G). The head 215 is oriented as shown (19H). The hex driver is rotated clockwise to tighten the screw until the head contacts the saddle (19I-J). The guide device 130 is inserted onto the tower, abutting the back surface of the temporary locking screw 190 (FIGS. 19K-L). Next, the second tower is inserted through the guide tube. (19M-N). The guide is removed and the hex driver is used to unscrew the temporary locking screw (19O). The locking screw is removed (19P) and the two towers are secured in the lateral mass (19Q).

In various embodiments, a fixation device 145, such as a polyaxial screw with tower feature, is used to provide additional stability to the spine following a spinal fusion procedure. The tower is an extension to a polyaxial screw, such as a pedicle screw or a lateral mass screw, that is used for minimally invasive posterior fixation systems as described herein. The tower may be a removable tower. The embodiments described above include a tower having a full length slot that permit a fixation rod to be lowered into place.

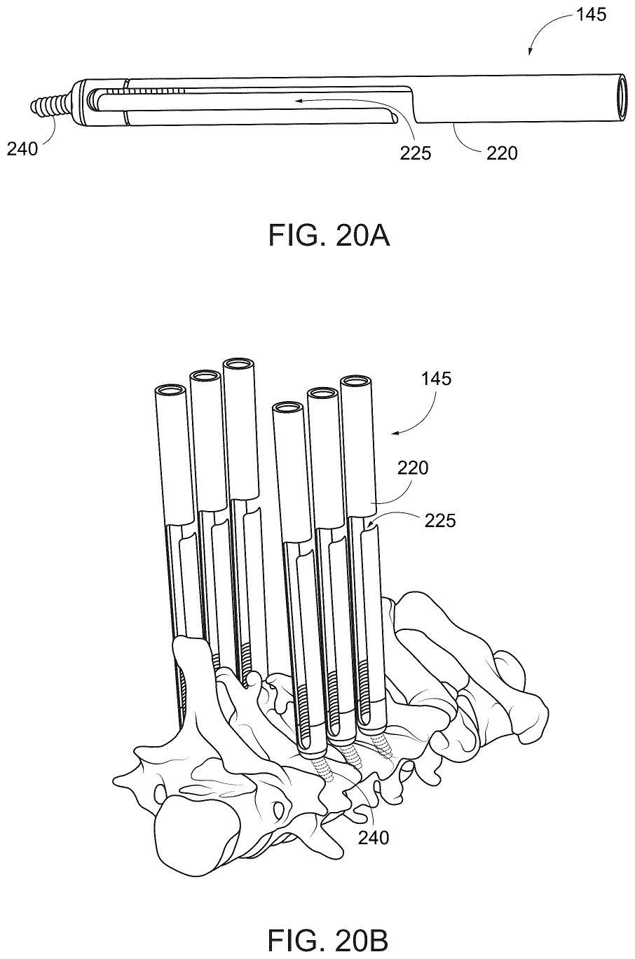

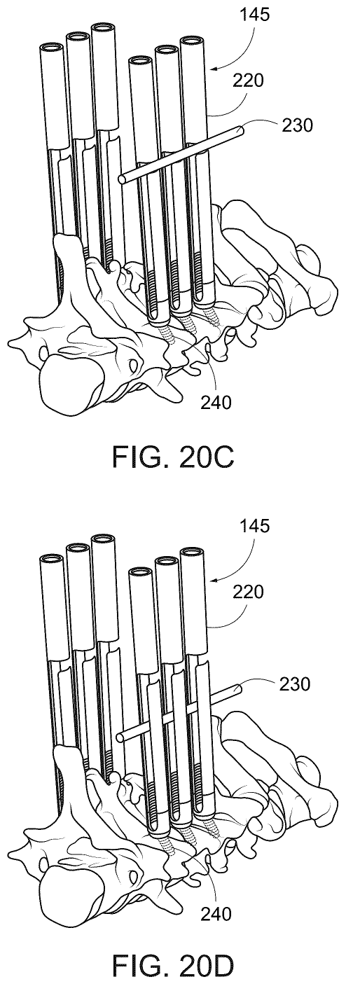

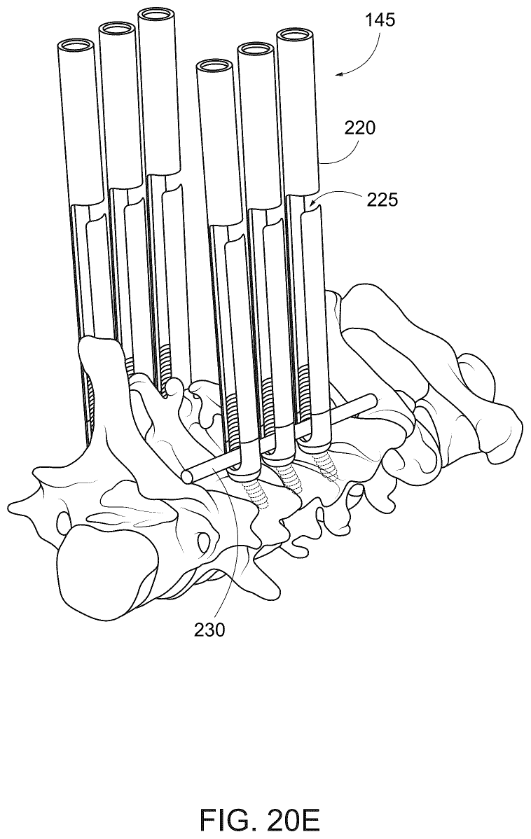

In another aspect, and as illustrated in FIGS. 20A-20E, a fixation device 145 for use with the devices and systems disclosed herein comprises an elongated tubular body or tower 220, a slot 225 and a screw 240. The slot 225 may be a partial slot or an L-shaped slot that extends at least part of the length of the tower and has a rod receiving opening formed in the outer circumference of the tower. The slot 225 receives a rod 230 for fixation of the screw 240 into the vertebrae. The slot extends along at least part of the length of the tower such that the rod is still inserted at a height above the skin level of the patient while still providing a solid cylindrical profile for at least part of the length of the tower above the slot entrance. In use, the fixation device 145 of FIG. 20A is implanted or deployed into the lateral mass of consecutive vertebrae and the slots 225 are lined up to receive the rod 230 (FIG. 20B). The fixation rod 230 is introduced into the slot (FIG. 20C) and advanced or slid down the slot (FIG. 20D) until it is seated in the screw heads (FIG. 20E). Set screws are used to secure the rod. Once secured, the excess length of the tower (“tower extension”) is cut or broken off while the fixation device remains in place.

All relative and directional references (including: upper, lower, upward, downward, left, right, leftward, rightward, top, bottom, side, above, below, front, middle, back, vertical, horizontal, and so forth) are given by way of example to aid the reader's understanding of the particular embodiments described herein. They should not be read to be requirements or limitations, particularly as to the position, orientation, or use unless specifically set forth in the claims. Connection references (e.g., attached, coupled, connected, joined, and the like) are to be construed broadly and may include intermediate members between a connection of elements and relative movement between elements. As such, connection references do not necessarily infer that two elements are directly connected and in fixed relation to each other, unless specifically set forth in the claims.

Although the invention has been disclosed in the context of certain embodiments and examples, the present invention extends beyond the specifically disclosed embodiments to other alternative embodiments and/or uses of the invention and obvious modifications and equivalents thereof. Thus, it is intended that the scope of the present invention herein disclosed should not be limited by the particular disclosed embodiments described above.

What is claimed is:

1. A spinal joint access device for accessing a spinal joint via a posterior access approach, the device comprising:
a body having a proximal portion and a distal portion; and
a chamfered or beveled end feature positioned at the distal portion of the body and configured for insertion at the spinal joint.
